(19) United States
(12) Patent Application Publication
(10) Pub. No.: US 2016/0040240 A1
(43) Pub. Date: Feb. 11, 2016

(54) CACNA1C ALLELE AND TREATMENT OF MOOD DISORDERS

(71) Applicant: THE UNIVERSITY OF CHICAGO, Chicago, IL (US)

(72) Inventors: Elliot Gershon, Chicago, IL (US); Chunyu Liu, Chicago, IL (US); Judith Badner, Chicago, IL (US)

(21) Appl. No.: 14/823,973

(22) Filed: Aug. 11, 2015

Related U.S. Application Data
(60) Provisional application No. 62/035,970, filed on Aug. 11, 2014.

(51) Int. Cl. C12Q 1/68; A61K 31/4439; A61K 31/4418; A61K 31/554; A61K 31/277; A61K 45/06

(52) U.S. Cl. CPC C12Q 1/6883; A61K 31/277; A61K 45/06; A61K 31/4418; A61K 31/554; A61K 31/4439; C12Q 2600/106; C12Q 2600/156

(57) ABSTRACT

Provided herein are methods of determining a treatment regimen for a subject with a mood disorder and methods of identifying a patient with a mood disorder as amenable to treatment with a calcium channel blocker (CCB). In exemplary embodiments, the methods comprise (a) analyzing a sample obtained from a subject with a mood disorder for the presence of allele [A] of CACNA1C, wherein allele [A] comprises the sequence of the polymorphic marker rs1006737.

### GRANT FUNDING

This invention was made with government support under Grant No. 1R01MH080425 awarded by the National Institutes of Health, and Grant No. 1R01MH094483-01 awarded by the National Institute of Mental Health. The government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 1,810,321 byte ACII (Text) file named “48562A_SeqListing.txt” created on Aug. 11, 2015.

### BACKGROUND

Bipolar I disorder (BD) is a serious mental illness with life threatening psychiatric morbidity and mortality. BD affects 1-2% of the general population and consumes a substantial portion of mental healthcare resources worldwide. It typically has a remitting and relapsing course that cycles between the mood extremes of mania and depression. Psychosis is common and 10-20% of patients become suicides.

Recent genetic association studies have shown a strong genetic association between BD and CACNA1C, one of the four possible L-type Calcium Channel alpha-1 subunit genes (see FIG. 1). The CACNA1C genetic risk allele is a functional regulator of expression of this subunit in one region in human brain [Gershon et al., Mol Psychiatry 19(8):890-4 (2013)]. Calcium channels are ubiquitous in synapses between excitable cells, including neurons and cardiac muscle cells (myocytes).

Calcium Channel Blockers (CCBs) were first discovered in 1964. CCBs affecting the L-type calcium channel continue to be used for their effects on the heart, as treatment for high blood pressure and for heart rhythm abnormalities. Clinicians soon noted that a substantial proportion of Bipolar patients treated with CCBs for cardiovascular disorder appeared to have improvement in their mood disorder. Published reports on treatment of BD with CCBs first appeared in 1982 [Caillard and Masse, Encephale 8:587-594 (1982)], and there were numerous reports over the following two decades, as reviewed by Cassamassima [Casamassima et al., Am J Med Genet B Neuropsychiatr Genet 153B: 1373-1393 (2010)].

While there have been multiple reports of improvement in manic patients treated with Verapamil and other CCBs, systematic trials demonstrating statistically significant effectiveness of CCBs in BD patients are lacking [Casamassima et al., 2010, supra].

### SUMMARY

The data from previous studies support that response to CCB therapy in BD patients varies. As supported by data presented herein, variability in patient response to CCB treatment is due to genetic differences among BD patients. More specifically, BD patients with the CACNA1C risk allele demonstrate reduced expression of the calcium channel gene and, BD patients who do not have the CACNA1C risk allele or have only one copy of the CACNA1C risk allele are, therefore, better responders to CCB treatment.

Provided herein are methods of treating a mood disorder in a subject. In exemplary embodiments, the method comprises (a) analyzing a sample obtained from the subject for the presence of an allele [A] of CACNA1C, wherein allele [A] comprises the sequence of polymorphic marker rs1006737; and (b) administering to the subject a calcium channel blocker (CCB) when no more than one copy of allele [A] is present in the cells of the sample.

Provided herein is a method of treating a mood disorder in a subject from which a sample was obtained, wherein the copy number of allele [A] of CACNA1C, comprising the sequence of polymorphic marker rs1006737, in the sample has been analyzed. Such a method comprises the step of administering to the subject an effective amount of a calcium channel blocker (CCB) when no more than one copy of the [A] allele is present in the cells of the sample.

A method of determining a treatment regimen for a subject with a mood disorder is further provided herein. In exemplary embodiments, the method comprises (a) analyzing a sample obtained from a subject with a mood disorder for the presence of allele [A] of CACNA1C, wherein allele [A] comprises the sequence of the polymorphic marker rs1006737; and (b) selecting a treatment regimen comprising administration of a calcium channel blocker (CCB), when no more than one copy of allele [A] is present in the cells of the sample.

A method of identifying a population of mood disorder patients amenable to treatment with a calcium channel blocker is also provided herein. In exemplary aspects, the method comprises (a) analyzing a sample obtained from a patient with a mood disorder for the presence of allele [A] of CACNA1C, wherein allele [A] comprises the sequence of the polymorphic marker rs1006737; and (b) identifying the patient as amenable to treatment with a calcium channel blocker when no more than one copy of allele [A] is present in the cells of the sample.

Related systems, e.g., computer systems, comprising a processor; a memory device coupled to the processor, and machine readable instructions stored on the memory device, are provided herein. In exemplary embodiments, the system comprises machine readable instructions that, when executed by the processor, cause the processor to: (a) receive a data value, a, that is a copy number determination of allele [A] of CACNA1C, wherein allele [A] comprises the sequence of polymorphic marker rs1006737, in the cells of a sample obtained from a subject; and (b) display an output relating to treating the subject for a mood disorder with a calcium channel blocker (CCB), when a is less than the diploid copy number of 2.

Related computer-readable storage media are also provided herein. In exemplary aspects, a computer-readable storage medium having stored thereon machine-readable instructions executable by a processor is provided. In exemplary aspects, the machine-readable instructions comprise: (a) instructions for receiving a data value, a, relating to the copy number of allele [A] of CACNA1C , wherein allele [A] comprises the sequence of the polymorphic marker rs1006737, in the cells of a sample obtained from a subject; and (b) instructions for displaying an output relating to treating the subject for a mood disorder with a calcium channel blocker (CCB), when a is less than the diploid copy number of 2.

Related methods implemented by a processor in a computer are provided herein. In exemplary embodiments, the method comprises (a) receiving a data value, a, relating to the copy number of allele [A] of CACNA1C, wherein allele [A] comprises the sequence of the polymorphic marker rs1006737, in the cells of a sample obtained from a subject; and (b) displaying an output relating to treating the subject for a mood disorder with a calcium channel blocker (CCB), when α is less than the diploid copy number of 2.

### BRIEF DESCRIPTION OF THE DRAWINGS

FIG. 1 is an illustration of an L-type calcium channel in the cell membrane. The L-type calcium channel controls the inward flow of calcium, an essential process in cell signaling. (Diagram from Wikipedia.)

FIG. 2 is an illustration of Timothy Syndrome type 2, functional mutations in Cav1.2, the protein product of CACNA1C. The mutation of the patient described in Example 1 is G4025. (Figure taken from Splawski et al., 2005.)

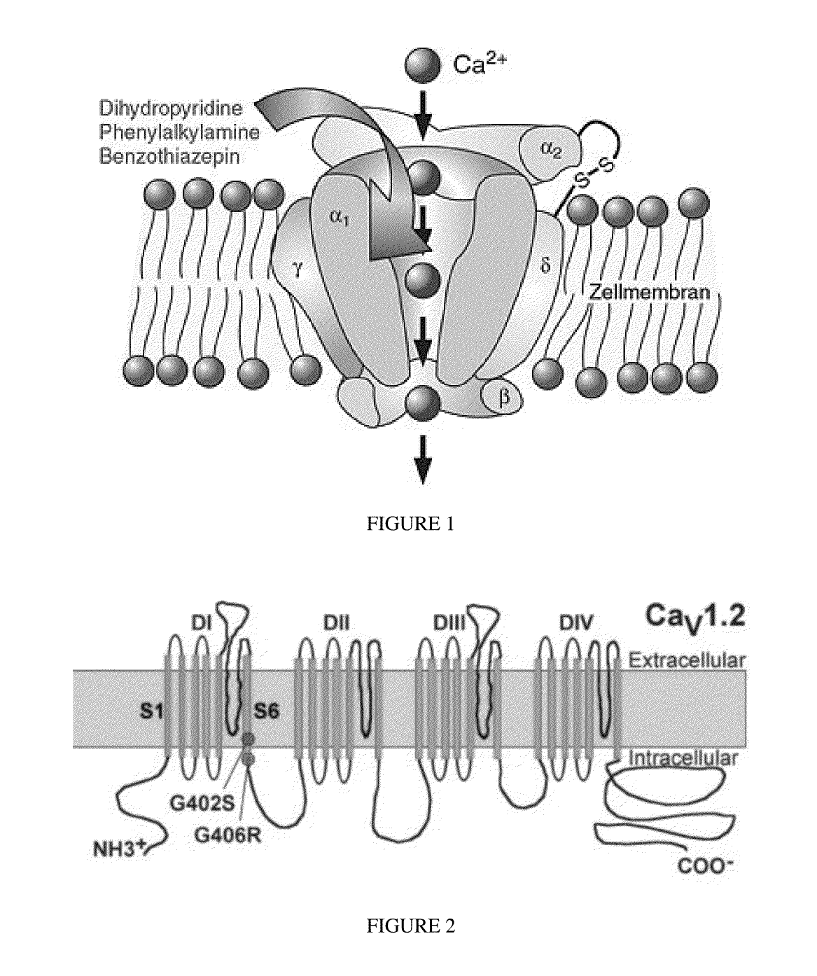

### DETAILED DESCRIPTION

Methods of Treating Mood Disorder

The invention provides methods of treating a mood disorder in a subject. In exemplary embodiments, the method comprises (a) analyzing a sample obtained from the subject for the presence of an allele [A] of CACNA1C, wherein allele [A] comprises the sequence of polymorphic marker rs1006737; and (b) administering to the subject a calcium channel blocker (CCB) when no more than one copy of allele [A] is present in the cells of the sample.

The invention also provides a method of treating a mood disorder in a subject from which a sample was obtained, wherein the copy number of allele [A] of CACNA1C, comprising the sequence of polymorphic marker rs1006737, in the sample has been analyzed. Such a method comprises the step of administering to the subject an effective amount of a calcium channel blocker (CCB) when no more than one copy of the [A] allele is present in the cells of the sample.

In exemplary aspects, the method of treating a mood disorder comprises administering to the subject a CCB when the subject is heterozygous for allele [A]. In exemplary aspects, the method of treating a mood disorder comprises administering to the subject a CCB when only one copy of the [A] allele is present in the cells of the sample. In exemplary aspects, the method of treating a mood disorder comprises administering to the subject a CCB when allele [A] is absent from the cells of the sample.

As used herein, the term “treat” as well as words stemming therefrom, e.g., “treating” and “treatment” do not necessarily imply 100% or complete treatment. Rather, there are varying degrees of treatment of which one of ordinary skill in the art recognizes as having a potential benefit or therapeutic effect. In this respect, the inventive methods can provide any amount of any level of treatment of the mood disorder in a subject. Furthermore, the treatment provided by the inventive method can include treatment of one or more conditions or symptoms of the disease, e.g., mood disorder, being treated.

Methods of Determining a Treatment Regimen for a Subject with Mood Disorder

The invention provides a method of determining a treatment regimen for a subject with a mood disorder is further provided herein. In exemplary embodiments, the method comprises (a) analyzing a sample obtained from a subject with a mood disorder for the presence of allele [A] of CACNA1C, wherein allele [A] comprises the sequence of the polymorphic marker rs1006737; and (b) selecting a treatment regimen comprising administration of a calcium channel blocker (CCB), when no more than one copy of allele [A] is present in the cells of the sample.

In exemplary aspects, the method of determining a treatment regimen for a subject with a mood disorder comprises selecting a treatment regimen comprising administration of a CCB, when the subject is heterozygous for the [A] allele. In exemplary aspects, the method of determining a treatment regimen for a subject with a mood disorder comprises selecting a treatment regimen comprising administration of a CCB, when only one copy of the [A] allele is present in the cells of the sample. In exemplary embodiments, the method of treating a mood disorder comprises selecting a treatment regimen comprising administration of a CCB, when allele [A] is absent from the sample.

Methods of Identifying a Population of Mood Disorder Patients Amenable to Treatment with a Calcium Channel Blocker

The invention also provides a method of identifying a population of mood disorder patients amenable to treatment with a calcium channel blocker is also provided herein. In exemplary aspects, the method comprises (a) analyzing a sample obtained from a patient with a mood disorder for the presence of allele [A] of CACNA1C, wherein allele [A] comprises the sequence of the polymorphic marker rs1006737; and (b) identifying the patient as amenable to treatment with a calcium channel blocker when no more than one copy of allele [A] is present in the cells of the sample.

The invention further provides a method of identifying a patient with a mood disorder as amenable to treatment with a calcium channel blocker is also provided herein. In exemplary aspects, the method comprises (a) analyzing a sample obtained from a patient with a mood disorder for the presence of allele [A] of CACNA1C, wherein allele [A] comprises the sequence of the polymorphic marker rs1006737; and (b) identifying the patient as amenable to treatment with a calcium channel blocker when no more than one copy of allele [A] is present in the cells of the sample.

In exemplary aspects, the method of identifying a population or a patient as amenable to treatment with a calcium channel blocker comprises identifying the patient as amenable to treatment with a calcium channel blocker, when the subject is heterozygous for the [A] allele. In exemplary aspects, the method of identifying a population or a patient as amenable to treatment with a calcium channel blocker comprises identifying the patient as amenable to treatment with a calcium channel blocker, when only one copy of the [A] allele is present in the cells of the sample. In exemplary embodiments, the method of identifying a population or a patient as amenable to treatment with a calcium channel blocker comprises identifying the patient as amenable to treatment with a calcium channel blocker, when allele [A] is absent from the sample.

Biomarkers and Measurement Thereof

In certain methods of the invention, a sample obtained from the subject with a mood disorder is analyzed for the presence of allele [A] of CACNA1C, or the copy number of allele [A] of CACNA1C, wherein allele [A] comprises the sequence of the polymorphic marker rs1006737. The CACNA1C gene is provided herein as SEQ ID NO: 1 and is publically available as National Center for Biotechnology Information (NCBI) Reference Sequence NG_{—}008801.2. The sequence of the polymorphic marker rs1006737 is provided herein as SEQ ID NO: 2. In SEQ ID NO: 2, the nucleotide at position 2282 is represented as “R” and, when R at position 2282 is G, the wild-type allele is represented, and, when R is A, the mutant risk allele is represented. The mutant risk allele, also referred to herein as “the risk allele” or “allele [A] of CACNA1C” or “allele [A]” comprises the sequence of SEQ ID NO: 3. In SEQ ID NO: 1, the polymorphic site is located at position 270344. SEQ ID NO: 1 includes the wild-type G at this position.

The sample may be analyzed for the presence of allele [A] or the copy number of allele [A] of CACNA1C by methods known in the art. In exemplary embodiments, the sample is analyzed for the sequence of CACNA1C. For example, the sample comprises saliva or cells, e.g., blood cells, neuronal cells, or brain cells, obtained from the subject, and the sequence of the genomic DNA of the cells is determined. In more particular aspects, the sequence of the CACNA1C gene, or a region thereof comprising the polymorphic site (i.e., position 2282 of SEQ ID NO: 2), of the genomic DNA of the saliva or cells (e.g., blood or neuronal cells) of the sample obtained from the subject is determined.

Suitable techniques of obtaining genomic sequence information from cells are known in the art. For example, the sequence data may be obtained through direct analysis of the sequence of the allele of the polymorphic marker. Suitable methods, some of which are described herein, include, for instance, whole genome analysis using a whole genome SNP chip (e.g., Infinium HD BeadChip), cloning for polymorphisms, non-radioactive PCR-single strand conformation polymorphism analysis, denaturing high pressure liquid chromatography (DHPLC), DNA hybridization, computational analysis, single-stranded conformational polymorphism (SSCP), restriction fragment length polymorphism (RFLP), automated fluorescent sequencing; clamped denaturing gel electrophoresis (CDGE); denaturing gradient gel electrophoresis (DGGE), mobility shift analysis, restriction enzyme analysis; heteroduplex analysis, chemical mismatch cleavage (CMC), RNase protection assays, use of polypeptides that recognize nucleotide mismatches, such as E. coli mutS protein, allele-specific PCR, and direct manual sequencing. These and other methods are described in the art (see, for instance, Li et al., Nucleic Acids Research, 28(2): e1 (i-v) (2000); Liu et al., Biochem Cell Bio 80:17-22 (2000); and Burczak et al., Polymorphism Detection and Analysis, Eaton Publishing, 2000; Sheffield et al., Proc. Natl. Acad. Sci. USA, 86:232-236 (1989); Orita et al., Proc. Natl. Acad. Sci. USA, 86:2766-2770 (1989); Flavell et al., Cell, 15:25-41 (1978); Geever et al., Proc. Natl. Acad. Sci. USA, 78:5081-5085 (1981); Cotton et al., Proc. Natl. Acad. Sci. USA, 85:4397-4401 (1985); Myers et al., Science 230:1242-1246 (1985); Church and Gilbert, Proc. Natl. Acad. Sci. USA, 81:1991-1995 (1988); Sanger et al., Proc. Natl. Acad. Sci. USA, 74:5463-5467 (1977); and Beavis et al., U.S. Pat. No. 5,288,644).

In exemplary embodiments, a hybridization method (see Current Protocols in Molecular Biology, Ausubel et al., eds., John Wiley & Sons, including all supplements) is used to analyze the sample for the presence (or absence) of allele [A]. A sample of genomic DNA, RNA, or cDNA is obtained from a subject. The DNA, RNA, or cDNA sample is then examined by incubating a sequence-specific nucleic acid probe with the DNA, RNA, or cDNA. A “nucleic acid probe”, as used herein, can be a DNA probe or an RNA probe that hybridizes to a complementary sequence. In exemplary aspects, the sequence-specific nucleic acid probe is designed to specifically hybridize to the sequence of allele [A]. One of skill in the art would know how to design such a probe so that sequence specific hybridization will occur only if a particular allele is present in a genomic sequence from a sample.

In exemplary aspects, the nucleic acid probe is conjugated to a detectable label. The detectable label, in exemplary aspects, is a radioisotope, a fluorophore, or an element particle. In a preferred embodiment, the DNA template containing the SNP polymorphism is amplified by Polymerase Chain Reaction (PCR) prior to detection.

Additional Steps

In exemplary aspects, the method may include additional steps. For example, the method may include repeating one or more of the recited step(s) of the method. In exemplary aspects, the methods comprises repeating the step of analyzing a sample obtained from the subject for the presence of an allele [A] of CACNA1C, wherein allele [A] comprises the sequence of polymorphic marker rs1006737. In alternative or additional aspects, the method of the invention comprises repeating the step of administering to the subject a CCB when no more than one copy of allele [A] is present in the cells of the sample.

In exemplary aspects, the repeated steps are carried out regularly. For example, the method may comprise analyzing a sample obtained from the subject for the presence of an allele [A] of CACNA1C daily, every 2 days, every 3 days, every 4 days, every 5 days, every 6 days, weekly, every 2 weeks, monthly, every 2 months, every 3 months, quarterly, bi-annually, or annually. Optionally, the sample is obtained from the subject at each instance.

In exemplary aspects, the method comprises analyzing the sample for CACNA1C expression. In exemplary aspects, the method comprises measuring the sample for a gene product encoded by CACNA1C. In exemplary aspects, the method comprises measuring the levels of RNA encoded by CACNA1C. In alternative or additional aspects, the method comprises measuring CACNA1C protein encoded by CACNA1C. In exemplary aspects, the method comprises measuring one or more CACNA1C isoforms, including but not limited to any of the following isoforms:

NCBI Reference Sequence for the mRNA → NCBI Reference Sequence for the
protein sequence, isoform name

NM_000719.6 (SEQ ID NO: 4)→ NP_000710.5 (SEQ ID NO: 5) voltage-dependent
L-type calcium channel subunit alpha-1C isoform 18
NM_001129827.1 (SEQ ID NO: 6)→ NP_001123299.1 (SEQ ID NO: 7) voltage-
dependent L-type calcium channel subunit alpha-1C isoform 2
NM_001129829.1 (SEQ ID NO: 8)→ NP_001123301.1 (SEQ ID NO: 9) voltage-
dependent L-type calcium channel subunit alpha-1C isoform 3
NM_001129830.2 (SEQ ID NO: 10)→ NP_001123302.2 (SEQ ID NO: 11) voltage-
dependent L-type calcium channel subunit alpha-1C isoform 4
NM_001129831.1 (SEQ ID NO: 12)→ NP_001123303.1 (SEQ ID NO: 13) voltage-
dependent L-type calcium channel subunit alpha-1C isoform 5
NM_001129832.1 (SEQ ID NO: 14)→ NP_001123304.1 (SEQ ID NO: 15) voltage-
dependent L-type calcium channel subunit alpha-1C isoform 6
NM_001129833.1 (SEQ ID NO: 16)→ NP_001123305.1 (SEQ ID NO: 17) voltage-
dependent L-type calcium channel subunit alpha-1C isoform 7
NM_001129834.1 (SEQ ID NO: 18)→ NP_001123306.1 (SEQ ID NO: 19) voltage-
dependent L-type calcium channel subunit alpha-1C isoform 8
NM_001129835.1 (SEQ ID NO: 20)→ NP_001123307.1 (SEQ ID NO: 21) voltage-
dependent L-type calcium channel subunit alpha-1C isoform 9
NM_001129836.1 (SEQ ID NO: 22)→ NP_001123308.1 (SEQ ID NO: 23) voltage-
dependent L-type calcium channel subunit alpha-1C isoform 10
NM_001129837.1 (SEQ ID NO: 24)→ NP_001123309.1 (SEQ ID NO: 25) voltage-
dependent L-type calcium channel subunit alpha-1C isoform 11
NM_001129838.1 (SEQ ID NO: 26)→ NP_001123310.1 (SEQ ID NO: 27) voltage-
dependent L-type calcium channel subunit alpha-1C isoform 12
NM_001129839.1 (SEQ ID NO: 28)→ NP_001123311.1 (SEQ ID NO: 29) voltage-
dependent L-type calcium channel subunit alpha-1C isoform 13
NM_001129840.1 (SEQ ID NO: 30)→ NP_001123312.1 (SEQ ID NO: 31) voltage-
dependent L-type calcium channel subunit alpha-1C isoform 14
NM_001129841.1 (SEQ ID NO: 32)→ NP_001123313.1 (SEQ ID NO: 33) voltage-
dependent L-type calcium channel subunit alpha-1C isoform 15
NM_001129842.1 (SEQ ID NO: 34)→ NP_001123314.1 (SEQ ID NO: 35) voltage-
dependent L-type calcium channel subunit alpha-1C isoform 16
NM_001129843.1 (SEQ ID NO: 36)→ NP_001123315.1 (SEQ ID NO: 37) voltage-
dependent L-type calcium channel subunit alpha-1C isoform 17
NM_001129844.1 (SEQ ID NO: 38)→ NP_001123316.1 (SEQ ID NO: 39) voltage-
dependent L-type calcium channel subunit alpha-1C isoform 19
NM_001129846.1 (SEQ ID NO: 40)→ NP_001123318.1 (SEQ ID NO: 41) voltage-
dependent L-type calcium channel subunit alpha-1C isoform 20
NM_001167623.1 (SEQ ID NO: 10)→ NP_001161095.1 (SEQ ID NO: 43) voltage-
dependent L-type calcium channel subunit alpha-1C isoform 21
NM_001167624.2 (SEQ ID NO: 10)→ NP_001161096.2 (SEQ ID NO: 45) voltage-
dependent L-type calcium channel subunit alpha-1C isoform 22
NM_001167625.1 (SEQ ID NO: 46)→ NP_001161097.1 (SEQ ID NO: 47) voltage-
dependent L-type calcium channel subunit alpha-1C isoform 23
NM_199460.3 (SEQ ID NO: 48)→ NP_955630.3 (SEQ ID NO: 49) voltage-
dependent L-type calcium channel subunit alpha-1C isoform 1

Methods of detecting RNA and proteins are known in the art and include, but not limited to, RT-PCR, qPCR, and RNA-Seq, Northern Blot, in situ hybridization, western blot, and ELISA. See, e.g., Sambrook et al., Molecular Cloning: A Laboratory Manual (Fourth Edition), Cold Spring Harbor Laboratory Press, Cold Spring Harbor, N.Y., 2012.

In exemplary aspects, wherein the method comprises measuring expression levels by measuring nucleic acids, e.g., RNA, mRNA, encoded by CACNA1C, the method further comprises amplifying at least a fragment of the nucleic acids to be measured. In exemplary aspects, the amplification is carried out via PCR or RT-PCR.

In exemplary aspects, the methods described herein further comprise genotyping the sample for additional polymorphic markers. In exemplary aspects, the method comprises genotyping the sample for one or more of the polymorphic markers listed in Table A. The human DNA sequences associated with these polymorphic markers are known in the art.

TABLE A

| | | | | Located within |
|---|---|---|---|---|
| SNP IDs | chr. | start | end | CACNA1C Gene |
| rs1000823 | chr12 | 3461461 | 3461462 | No |
| rs1001127 | chr12 | 6601742 | 6601743 | No |
| rs1005375 | chr12 | 3436758 | 3436759 | No |
| rs1006696 | chr12 | 3405104 | 3405105 | No |
| rs1006737 | chr12 | 2345294 | 2345295 | Yes |
| rs10153570 | chr2 | 131708648 | 131708649 | No |
| rs1015832 | chr12 | 5690229 | 5690230 | No |
| rs10160993 | chr12 | 2499564 | 2499565 | Yes |
| rs1016388 | chr12 | 2321867 | 2321868 | Yes |
| rs1017101 | chr12 | 6574300 | 6574301 | No |
| rs10219509 | chr12 | 4300808 | 4300809 | No |
| rs1024582 | chr12 | 2402245 | 2402246 | Yes |
| rs1034969 | chr12 | 6573855 | 6573856 | No |
| rs1043262 | chr12 | 6639087 | 6639088 | No |
| rs10437827 | chr12 | 4473328 | 4473329 | No |
| rs10459125 | chr12 | 2309863 | 2309864 | Yes |
| rs10459134 | chr12 | 5879850 | 5879851 | No |
| rs10466913 | chr12 | 6022494 | 6022495 | No |
| rs10491954 | chr12 | 1354344 | 1354345 | No |
| rs10491961 | chr12 | 2115709 | 2115710 | No |
| rs10491969 | chr12 | 3734335 | 3734336 | No |
| rs10492061 | chr12 | 4991284 | 4991285 | No |
| rs10492094 | chr12 | 5478147 | 5478148 | No |
| rs10492097 | chr12 | 6394677 | 6394678 | No |
| rs10492184 | chr12 | 5699047 | 5699048 | No |
| rs10505724 | chr12 | 2782069 | 2782070 | Yes |
| rs1056008 | chr12 | 662837 | 662838 | No |
| rs1060499 | chr12 | 1020265 | 1020266 | No |
| rs1063856 | chr12 | 6153533 | 6153534 | No |
| rs1063857 | chr12 | 6153513 | 6153514 | No |
| rs10735005 | chr12 | 1995402 | 1995403 | No |
| rs10735020 | chr12 | 3414983 | 3414984 | No |
| rs10735026 | chr12 | 3738848 | 3738849 | No |
| rs10744559 | chr12 | 2298297 | 2298298 | Yes |
| rs10744560 | chr12 | 2387098 | 2387099 | Yes |
| rs10744601 | chr12 | 3405901 | 3405902 | No |
| rs10744641 | chr12 | 4436832 | 4436833 | No |
| rs10744648 | chr12 | 586406 | 586407 | No |
| rs10744675 | chr12 | 5145121 | 5145122 | No |
| rs10744700 | chr12 | 6297862 | 6297863 | No |
| rs1076344 | chr12 | 2553987 | 2553988 | Yes |
| rs1076346 | chr12 | 2554277 | 2554278 | Yes |
| rs10773979 | chr12 | 1788863 | 1788864 | No |
| rs10774029 | chr12 | 2280806 | 2280807 | Yes |
| rs10774030 | chr12 | 2292689 | 2292690 | Yes |
| rs10774033 | chr12 | 2314425 | 2314426 | Yes |
| rs10774034 | chr12 | 2330457 | 2330458 | Yes |
| rs10774035 | chr12 | 2368673 | 2368674 | Yes |
| rs10774036 | chr12 | 2386947 | 2386948 | Yes |
| rs10774037 | chr12 | 2420525 | 2420526 | Yes |
| rs10774117 | chr12 | 3219859 | 3219860 | No |
| rs10774120 | chr12 | 3224985 | 3224986 | No |
| rs10774140 | chr12 | 3461601 | 3461602 | No |
| rs10774148 | chr12 | 3557319 | 3557320 | No |
| rs10774152 | chr12 | 495216 | 495217 | No |
| rs10774156 | chr12 | 3664703 | 3664704 | No |
| rs10774167 | chr12 | 3844380 | 3844381 | No |
| rs10774168 | chr12 | 3846298 | 3846299 | No |
| rs10774169 | chr12 | 516345 | 516346 | No |
| rs10774224 | chr12 | 4471634 | 4471635 | No |
| rs10774232 | chr12 | 4518168 | 4518169 | No |
| rs10774250 | chr12 | 4735427 | 4735428 | No |
| rs10774324 | chr12 | 5476396 | 5476397 | No |
| rs10774325 | chr12 | 5476904 | 5476905 | No |
| rs10774333 | chr12 | 5563166 | 5563167 | No |
| rs10774334 | chr12 | 5611798 | 5611799 | No |
| rs10774353 | chr12 | 5767173 | 5767174 | No |
| rs10774376 | chr12 | 5941323 | 5941324 | No |
| rs10774403 | chr12 | 6277310 | 6277311 | No |
| rs10774405 | chr12 | 6285390 | 6285391 | No |
| rs10774466 | chr12 | 939301 | 939302 | No |
| rs10848438 | chr12 | 1211575 | 1211576 | No |
| rs10848476 | chr12 | 1587159 | 1587160 | No |
| rs10848587 | chr12 | 2002361 | 2002362 | No |
| rs10848596 | chr12 | 2034886 | 2034887 | No |
| rs10848600 | chr12 | 2083927 | 2083928 | No |
| rs10848622 | chr12 | 2274050 | 2274051 | Yes |
| rs10848623 | chr12 | 359462 | 359463 | No |
| rs10848627 | chr12 | 2312407 | 2312408 | Yes |
| rs10848628 | chr12 | 2312488 | 2312489 | Yes |
| rs10848629 | chr12 | 2312896 | 2312897 | Yes |
| rs10848632 | chr12 | 2315992 | 2315993 | Yes |
| rs10848633 | chr12 | 2316018 | 2316019 | Yes |
| rs10848634 | chr12 | 2316126 | 2316127 | Yes |
| rs10848635 | chr12 | 2316194 | 2316195 | Yes |
| rs10848636 | chr12 | 2316492 | 2316493 | Yes |
| rs10848637 | chr12 | 2316553 | 2316554 | Yes |
| rs10848642 | chr12 | 2331571 | 2331572 | Yes |
| rs10848644 | chr12 | 365288 | 365289 | No |
| rs10848645 | chr12 | 2420243 | 2420244 | Yes |
| rs10848649 | chr12 | 2460085 | 2460086 | Yes |
| rs10848683 | chr12 | 2791129 | 2791130 | Yes |
| rs10848703 | chr12 | 2882249 | 2882250 | No |
| rs10848784 | chr12 | 3188513 | 3188514 | No |
| rs10848789 | chr12 | 3195201 | 3195202 | No |
| rs10848803 | chr12 | 3222755 | 3222756 | No |
| rs10848818 | chr12 | 3293243 | 3293244 | No |
| rs10848821 | chr12 | 3298030 | 3298031 | No |
| rs10848863 | chr12 | 3561809 | 3561810 | No |
| rs10848895 | chr12 | 3749311 | 3749312 | No |
| rs10848905 | chr12 | 507788 | 507789 | No |
| rs10848914 | chr12 | 3844094 | 3844095 | No |
| rs10848918 | chr12 | 3853328 | 3853329 | No |
| rs10849017 | chr12 | 4322050 | 4322051 | No |
| rs10849038 | chr12 | 4437378 | 4437379 | No |
| rs10849059 | chr12 | 4521663 | 4521664 | No |
| rs10849087 | chr12 | 4650064 | 4650065 | No |
| rs10849168 | chr12 | 4986995 | 4986996 | No |
| rs10849175 | chr12 | 5030706 | 5030707 | No |
| rs10849216 | chr12 | 5322961 | 5322962 | No |
| rs10849222 | chr12 | 5369482 | 5369483 | No |
| rs10849261 | chr12 | 5522555 | 5522556 | No |
| rs10849265 | chr12 | 5532011 | 5532012 | No |
| rs10849290 | chr12 | 5663171 | 5663172 | No |
| rs10849298 | chr12 | 5691999 | 5692000 | No |
| rs10849310 | chr12 | 5752724 | 5752725 | No |
| rs10849401 | chr12 | 6273237 | 6273238 | No |
| rs10849414 | chr12 | 6286369 | 6286370 | No |
| rs10849573 | chr12 | 982320 | 982321 | No |
| rs10849575 | chr12 | 230403 | 230404 | No |
| rs11061839 | chr12 | 1655448 | 1655449 | No |
| rs11062012 | chr12 | 1982124 | 1982125 | No |
| rs11062038 | chr12 | 2089229 | 2089230 | No |
| rs11062040 | chr12 | 2091256 | 2091257 | No |
| rs11062055 | chr12 | 2107300 | 2107301 | No |
| rs11062057 | chr12 | 2114605 | 2114606 | No |
| rs11062068 | chr12 | 2121723 | 2121724 | No |
| rs11062114 | chr12 | 2198826 | 2198827 | Yes |
| rs11062145 | chr12 | 2297352 | 2297353 | Yes |
| rs11062156 | chr12 | 2317522 | 2317523 | Yes |
| rs11062159 | chr12 | 2326223 | 2326224 | Yes |
| rs11062161 | chr12 | 2329969 | 2329970 | Yes |
| rs11062162 | chr12 | 2332103 | 2332104 | Yes |
| rs11062170 | chr12 | 2348843 | 2348844 | Yes |
| rs11062196 | chr12 | 2460106 | 2460107 | Yes |
| rs11062197 | chr12 | 2460387 | 2460388 | Yes |
| rs11062290 | chr12 | 2745150 | 2745151 | Yes |
| rs11062293 | chr12 | 2748274 | 2748275 | Yes |
| rs11062309 | chr12 | 2784987 | 2784988 | Yes |
| rs11062351 | chr12 | 2881762 | 2881763 | No |
| rs11062385 | chr12 | 427574 | 427575 | No |
| rs11062630 | chr12 | 3474413 | 3474414 | No |
| rs11062739 | chr12 | 3711361 | 3711362 | No |
| rs11062745 | chr12 | 3724582 | 3724583 | No |
| rs11062747 | chr12 | 3750108 | 3750109 | No |
| rs11062752 | chr12 | 3757387 | 3757388 | No |
| rs11062760 | chr12 | 3788528 | 3788529 | No |
| rs11062770 | chr12 | 3822839 | 3822840 | No |
| rs11062772 | chr12 | 3826642 | 3826643 | No |
| rs11062776 | chr12 | 3831992 | 3831993 | No |
| rs11062778 | chr12 | 3834326 | 3834327 | No |
| rs11062885 | chr12 | 3978660 | 3978661 | No |
| rs11062886 | chr12 | 3980955 | 3980956 | No |
| rs11062932 | chr12 | 4064155 | 4064156 | No |
| rs11063022 | chr12 | 4296516 | 4296517 | No |
| rs11063027 | chr12 | 4299312 | 4299313 | No |
| rs11063069 | chr12 | 4374372 | 4374373 | No |
| rs11063099 | chr12 | 4462504 | 4462505 | No |
| rs11063101 | chr12 | 4466402 | 4466403 | No |
| rs11063182 | chr12 | 4581370 | 4581371 | No |
| rs11063189 | chr12 | 4588247 | 4588248 | No |
| rs11063199 | chr12 | 4591853 | 4591854 | No |
| rs11063205 | chr12 | 4597744 | 4597745 | No |
| rs11063207 | chr12 | 4603697 | 4603698 | No |
| rs11063282 | chr12 | 4760977 | 4760978 | No |
| rs11063426 | chr12 | 5065592 | 5065593 | No |
| rs11063462 | chr12 | 5121112 | 5121113 | No |
| rs11063468 | chr12 | 5135473 | 5135474 | No |
| rs11063476 | chr12 | 5140981 | 5140982 | No |
| rs11063490 | chr12 | 5191130 | 5191131 | No |
| rs11063539 | chr12 | 658113 | 658114 | No |
| rs11063567 | chr12 | 5322550 | 5322551 | No |
| rs11063623 | chr12 | 5453278 | 5453279 | No |
| rs11063634 | chr12 | 5470168 | 5470169 | No |
| rs11063663 | chr12 | 5525569 | 5525570 | No |
| rs11063692 | chr12 | 5571844 | 5571845 | No |
| rs11063806 | chr12 | 708596 | 708597 | No |
| rs11063995 | chr12 | 6136152 | 6136153 | No |
| rs11064020 | chr12 | 6196313 | 6196314 | No |
| rs11064021 | chr12 | 6196412 | 6196413 | No |
| rs11064024 | chr12 | 6202048 | 6202049 | No |
| rs11064153 | chr12 | 6488449 | 6488450 | No |
| rs11064294 | chr12 | 805450 | 805451 | No |
| rs11064562 | chr12 | 945321 | 945322 | No |
| rs1108073 | chr12 | 2333637 | 2333638 | Yes |
| rs1108074 | chr12 | 2333483 | 2333484 | Yes |
| rs1108221 | chr12 | 2332794 | 2332795 | Yes |
| rs1108222 | chr12 | 2332855 | 2332856 | Yes |
| rs1108385 | chr12 | 2482329 | 2482330 | Yes |
| rs11559856 | chr12 | 5850903 | 5850904 | No |
| rs11609462 | chr12 | 1590068 | 1590069 | No |
| rs11609728 | chr12 | 6139691 | 6139692 | No |
| rs11609815 | chr12 | 6134230 | 6134231 | No |
| rs11610204 | chr12 | 3510916 | 3510917 | No |
| rs11610629 | chr12 | 6137455 | 6137456 | No |
| rs11611163 | chr12 | 4279482 | 4279483 | No |
| rs11611543 | chr12 | 5476043 | 5476044 | No |
| rs11611917 | chr12 | 6136633 | 6136634 | No |
| rs11612401 | chr12 | 6136967 | 6136968 | No |
| rs11612864 | chr12 | 2462222 | 2462223 | Yes |
| rs11613331 | chr12 | 351466 | 351467 | No |
| rs11613469 | chr12 | 2074021 | 2074022 | No |
| rs11613749 | chr12 | 3708498 | 3708499 | No |
| rs11614030 | chr12 | 4769875 | 4769876 | No |
| rs11614912 | chr12 | 6148554 | 6148555 | No |
| rs11615941 | chr12 | 3556798 | 3556799 | No |
| rs11616050 | chr12 | 783349 | 783350 | No |
| rs11616055 | chr12 | 3849592 | 3849593 | No |
| rs11616162 | chr12 | 4591080 | 4591081 | No |
| rs11677224 | chr2 | 88789814 | 88789815 | No |
| rs11835708 | chr12 | 3515553 | 3515554 | No |
| rs12191018 | chr6 | 153479725 | 153479726 | No |
| rs12230555 | chr12 | 4418783 | 4418784 | No |
| rs12303498 | chr12 | 3411437 | 3411438 | No |
| rs12304321 | chr12 | 5163047 | 5163048 | No |
| rs12304385 | chr12 | 5898503 | 5898504 | No |
| rs12315711 | chr12 | 2346829 | 2346830 | Yes |
| rs12320409 | chr12 | 3428427 | 3428428 | No |
| rs12368779 | chr12 | 1982150 | 1982151 | No |
| rs12369414 | chr12 | 915769 | 915770 | No |
| rs12369484 | chr12 | 6271972 | 6271973 | No |
| rs12370980 | chr12 | 3849483 | 3849484 | No |
| rs12422231 | chr12 | 3803565 | 3803566 | No |
| rs12422554 | chr12 | 2312619 | 2312620 | Yes |
| rs12423086 | chr12 | 1344140 | 1344141 | No |
| rs12423234 | chr12 | 4930359 | 4930360 | No |
| rs12423277 | chr12 | 2314318 | 2314319 | Yes |
| rs12423361 | chr12 | 3657685 | 3657686 | No |
| rs12423920 | chr12 | 5047775 | 5047776 | No |
| rs12424245 | chr12 | 2322512 | 2322513 | Yes |
| rs12425157 | chr12 | 1357859 | 1357860 | No |
| rs12426185 | chr12 | 5709634 | 5709635 | No |
| rs12470817 | chr2 | 131703693 | 131703694 | No |
| rs12473388 | chr2 | 45549304 | 45549305 | No |
| rs12579294 | chr12 | 3289944 | 3289945 | No |
| rs12580433 | chr12 | 5800575 | 5800576 | No |
| rs12811453 | chr12 | 4761010 | 4761011 | No |
| rs12816718 | chr12 | 969799 | 969800 | No |
| rs12817188 | chr12 | 5567618 | 5567619 | No |
| rs12818111 | chr12 | 3501260 | 3501261 | No |
| rs12823931 | chr12 | 4467920 | 4467921 | No |
| rs12825233 | chr12 | 5453776 | 5453777 | No |
| rs12830185 | chr12 | 5101463 | 5101464 | No |
| rs13395344 | chr2 | 88795167 | 88795168 | No |
| rs1355685 | chr3 | 145892710 | 145892711 | No |
| rs1420402 | chr12 | 5377547 | 5377548 | No |
| rs1421181 | chr18 | 43068785 | 43068786 | No |
| rs1468765 | chr12 | 4296893 | 4296894 | No |
| rs1532609 | chr12 | 232122 | 232123 | No |
| rs1544502 | chr12 | 2438809 | 2438810 | Yes |
| rs1544514 | chr12 | 2558185 | 2558186 | Yes |
| rs1544608 | chr12 | 5813533 | 5813534 | No |
| rs1548904 | chr12 | 372381 | 372382 | No |
| rs1558142 | chr12 | 2022410 | 2022411 | No |
| rs1558321 | chr12 | 2230319 | 2230320 | Yes |
| rs1558322 | chr12 | 2230054 | 2230055 | Yes |
| rs1558325 | chr12 | 6289107 | 6289108 | No |
| rs1558506 | chr12 | 5750895 | 5750896 | No |
| rs1558507 | chr12 | 5747821 | 5747822 | No |
| rs1558508 | chr12 | 5743213 | 5743214 | No |
| rs1610056 | chr12 | 6195396 | 6195397 | No |
| rs16928082 | chr12 | 6396155 | 6396156 | No |
| rs16928108 | chr12 | 989396 | 989397 | No |
| rs16930578 | chr12 | 3691436 | 3691437 | No |
| rs16931965 | chr12 | 976486 | 976487 | No |
| rs16932084 | chr12 | 1080853 | 1080854 | No |
| rs16933825 | chr12 | 5842890 | 5842891 | No |
| rs17178877 | chr12 | 5175402 | 5175403 | No |
| rs17179631 | chr12 | 5295432 | 5295433 | No |
| rs17179798 | chr12 | 5314507 | 5314508 | No |
| rs17722632 | chr12 | 5571980 | 5571981 | No |
| rs17754970 | chr12 | 773261 | 773262 | No |
| rs17769092 | chr12 | 3464296 | 3464297 | No |
| rs17769954 | chr12 | 3699213 | 3699214 | No |
| rs17770663 | chr12 | 3791142 | 3791143 | No |
| rs17778793 | chr12 | 5444246 | 5444247 | No |
| rs17778948 | chr12 | 5478112 | 5478113 | No |
| rs17835992 | chr12 | 3668988 | 3668989 | No |
| rs1800380 | chr12 | 6138594 | 6138595 | No |
| rs1808285 | chr12 | 3852359 | 3852360 | No |
| rs1860002 | chr12 | 2413802 | 2413803 | Yes |
| rs1860004 | chr12 | 2477048 | 2477049 | Yes |
| rs1860048 | chr12 | 2058694 | 2058695 | No |
| rs1860053 | chr12 | 1964415 | 1964416 | No |
| rs1860056 | chr12 | 2311835 | 2311836 | Yes |
| rs1860363 | chr12 | 6276532 | 6276533 | No |
| rs1860365 | chr12 | 6147109 | 6147110 | No |
| rs1860431 | chr12 | 3183026 | 3183027 | No |
| rs1860449 | chr12 | 3674830 | 3674831 | No |
| rs1861584 | chr12 | 5707817 | 5707818 | No |
| rs1972582 | chr12 | 374301 | 374302 | No |
| rs2007044 | chr12 | 2344959 | 2344960 | Yes |
| rs2008134 | chr12 | 6588444 | 6588445 | No |
| rs2010932 | chr12 | 2085981 | 2085982 | No |
| rs2013064 | chr12 | 1998043 | 1998044 | No |
| rs2013195 | chr12 | 3840420 | 3840421 | No |
| rs2016141 | chr12 | 5146002 | 5146003 | No |
| rs2017273 | chr12 | 2100022 | 2100023 | No |
| rs2041135 | chr12 | 2101497 | 2101498 | No |
| rs2058349 | chr12 | 3844595 | 3844596 | No |
| rs2058468 | chr12 | 5819953 | 5819954 | No |
| rs2072373 | chr12 | 6631887 | 6631888 | No |
| rs2072374 | chr12 | 6637844 | 6637845 | No |
| rs2075031 | chr12 | 669037 | 669038 | No |
| rs2075316 | chr12 | 4492805 | 4492806 | No |
| rs2075317 | chr12 | 4492314 | 4492315 | No |
| rs2079867 | chr12 | 6622110 | 6622111 | No |
| rs2079868 | chr12 | 6622345 | 6622346 | No |
| rs2080105 | chr12 | 5780595 | 5780596 | No |
| rs2107613 | chr12 | 888427 | 888428 | No |
| rs2108570 | chr12 | 2421587 | 2421588 | Yes |
| rs2108638 | chr12 | 2065369 | 2065370 | No |
| rs2108639 | chr12 | 1973383 | 1973384 | No |
| rs2109112 | chr12 | 3987472 | 3987473 | No |
| rs2109425 | chr12 | 4977334 | 4977335 | No |
| rs2109426 | chr12 | 6589521 | 6589522 | No |
| rs2158502 | chr12 | 936467 | 936468 | No |
| rs2159100 | chr12 | 2346392 | 2346393 | Yes |
| rs2159407 | chr12 | 3186923 | 3186924 | No |
| rs2159410 | chr12 | 3112728 | 3112729 | No |
| rs216011 | chr12 | 2725617 | 2725618 | Yes |
| rs216014 | chr12 | 2730450 | 2730451 | Yes |
| rs216043 | chr12 | 2760343 | 2760344 | Yes |
| rs216046 | chr12 | 2764000 | 2764001 | Yes |
| rs216325 | chr12 | 6145528 | 6145529 | No |
| rs216327 | chr12 | 6146148 | 6146149 | No |
| rs216329 | chr12 | 6146506 | 6146507 | No |
| rs216334 | chr12 | 6147610 | 6147611 | No |
| rs216855 | chr12 | 6085512 | 6085513 | No |
| rs216856 | chr12 | 6086264 | 6086265 | No |
| rs216865 | chr12 | 6090302 | 6090303 | No |
| rs216867 | chr12 | 6090999 | 6091000 | No |
| rs216873 | chr12 | 6095273 | 6095274 | No |
| rs2189669 | chr12 | 1586845 | 1586846 | No |
| rs2190769 | chr12 | 2008798 | 2008799 | No |
| rs2190770 | chr12 | 2014352 | 2014353 | No |
| rs2191365 | chr12 | 5032639 | 5032640 | No |
| rs2191537 | chr12 | 3851709 | 3851710 | No |
| rs2215738 | chr12 | 5784873 | 5784874 | No |
| rs2229351 | chr12 | 406291 | 406292 | No |
| rs2238022 | chr12 | 2186477 | 2186478 | Yes |
| rs2238023 | chr12 | 2202536 | 2202537 | Yes |
| rs2238027 | chr12 | 2216957 | 2216958 | Yes |
| rs2238029 | chr12 | 2218895 | 2218896 | Yes |
| rs2238043 | chr12 | 2275662 | 2275663 | Yes |
| rs2238046 | chr12 | 2299997 | 2299998 | Yes |
| rs2238048 | chr12 | 2300206 | 2300207 | Yes |
| rs2238049 | chr12 | 2306127 | 2306128 | Yes |
| rs2238050 | chr12 | 2306706 | 2306707 | Yes |
| rs2238052 | chr12 | 2307295 | 2307296 | Yes |
| rs2238053 | chr12 | 2317095 | 2317096 | Yes |
| rs2238054 | chr12 | 2317643 | 2317644 | Yes |
| rs2238056 | chr12 | 2327943 | 2327944 | Yes |
| rs2238057 | chr12 | 2384004 | 2384005 | Yes |
| rs2238069 | chr12 | 2456061 | 2456062 | Yes |
| rs2238070 | chr12 | 2456114 | 2456115 | Yes |
| rs2238077 | chr12 | 2466083 | 2466084 | Yes |
| rs2238078 | chr12 | 2472451 | 2472452 | Yes |
| rs2238079 | chr12 | 2473357 | 2473358 | Yes |
| rs2238083 | chr12 | 2487000 | 2487001 | Yes |
| rs2238090 | chr12 | 2683331 | 2683332 | Yes |
| rs2238103 | chr12 | 6194182 | 6194183 | No |
| rs2238104 | chr12 | 6187664 | 6187665 | No |
| rs2239017 | chr12 | 2289650 | 2289651 | Yes |
| rs2239018 | chr12 | 2304356 | 2304357 | Yes |
| rs2239019 | chr12 | 2310309 | 2310310 | Yes |
| rs2239023 | chr12 | 2310615 | 2310616 | Yes |
| rs2239030 | chr12 | 2335940 | 2335941 | Yes |
| rs2239032 | chr12 | 2337137 | 2337138 | Yes |
| rs2239033 | chr12 | 2337745 | 2337746 | Yes |
| rs2239037 | chr12 | 2363715 | 2363716 | Yes |
| rs2239038 | chr12 | 2374129 | 2374130 | Yes |
| rs2239047 | chr12 | 2435705 | 2435706 | Yes |
| rs2239061 | chr12 | 2502511 | 2502512 | Yes |
| rs2239062 | chr12 | 2502571 | 2502572 | Yes |
| rs2239074 | chr12 | 2538548 | 2538549 | Yes |
| rs2239079 | chr12 | 2550817 | 2550818 | Yes |
| rs2239080 | chr12 | 2554688 | 2554689 | Yes |
| rs2239093 | chr12 | 2609561 | 2609562 | Yes |
| rs2239131 | chr12 | 2229058 | 2229059 | Yes |
| rs2239144 | chr12 | 6196182 | 6196183 | No |
| rs2239145 | chr12 | 6195995 | 6195996 | No |
| rs2239147 | chr12 | 6195526 | 6195527 | No |
| rs2240283 | chr12 | 966123 | 966124 | No |
| rs2240610 | chr12 | 2057591 | 2057592 | No |
| rs2240612 | chr12 | 2058244 | 2058245 | No |
| rs2255390 | chr12 | 999761 | 999762 | No |
| rs2270152 | chr12 | 6061068 | 6061069 | No |
| rs2283275 | chr12 | 2184559 | 2184560 | Yes |
| rs2283276 | chr12 | 2184846 | 2184847 | Yes |
| rs2283287 | chr12 | 2298723 | 2298724 | Yes |
| rs2283288 | chr12 | 2299047 | 2299048 | Yes |
| rs2283291 | chr12 | 2337459 | 2337460 | Yes |
| rs2283294 | chr12 | 2423619 | 2423620 | Yes |
| rs2283301 | chr12 | 2446805 | 2446806 | Yes |
| rs2283304 | chr12 | 2473970 | 2473971 | Yes |
| rs2283306 | chr12 | 2489389 | 2489390 | Yes |
| rs2286006 | chr12 | 968488 | 968489 | No |
| rs2286374 | chr12 | 1962758 | 1962759 | No |
| rs2286646 | chr12 | 6061490 | 6061491 | No |
| rs2291094 | chr12 | 5294805 | 5294806 | No |
| rs2291095 | chr12 | 5294346 | 5294347 | No |
| rs2291919 | chr12 | 280348 | 280349 | No |
| rs2291920 | chr12 | 278375 | 278376 | No |
| rs2291922 | chr12 | 275064 | 275065 | No |
| rs2299656 | chr12 | 2165146 | 2165147 | Yes |
| rs2299664 | chr12 | 2226044 | 2226045 | Yes |
| rs2300134 | chr12 | 478854 | 478855 | No |
| rs2301880 | chr12 | 1003836 | 1003837 | No |
| rs2302729 | chr12 | 2783971 | 2783972 | Yes |
| rs2302840 | chr18 | 43072986 | 43072987 | No |
| rs2335817 | chr12 | 3294235 | 3294236 | No |
| rs2361590 | chr12 | 5775386 | 5775387 | No |
| rs2363877 | chr12 | 6292460 | 6292461 | No |
| rs2363878 | chr12 | 6292974 | 6292975 | No |
| rs2363880 | chr12 | 6302008 | 6302009 | No |
| rs2369276 | chr12 | 486172 | 486173 | No |
| rs2369280 | chr12 | 508858 | 508859 | No |
| rs2369703 | chr12 | 1333716 | 1333717 | No |
| rs2370413 | chr12 | 2354869 | 2354870 | Yes |
| rs2370414 | chr12 | 2368395 | 2368396 | Yes |
| rs2370515 | chr12 | 2511161 | 2511162 | Yes |
| rs2370596 | chr12 | 2666347 | 2666348 | Yes |
| rs240826 | chr12 | 5324763 | 5324764 | No |
| rs241966 | chr12 | 3839964 | 3839965 | No |
| rs241970 | chr12 | 3838416 | 3838417 | No |
| rs241975 | chr12 | 3836655 | 3836656 | No |
| rs241978 | chr12 | 3834823 | 3834824 | No |
| rs241982 | chr12 | 3831610 | 3831611 | No |
| rs241983 | chr12 | 3831159 | 3831160 | No |
| rs242012 | chr12 | 3790146 | 3790147 | No |
| rs2429123 | chr12 | 2113146 | 2113147 | No |
| rs2429127 | chr12 | 2116767 | 2116768 | No |
| rs2429135 | chr12 | 2124954 | 2124955 | No |
| rs2429159 | chr12 | 2016321 | 2016322 | No |
| rs2429163 | chr12 | 2022500 | 2022501 | No |
| rs2429175 | chr12 | 2045084 | 2045085 | No |
| rs2470395 | chr12 | 2145297 | 2145298 | No |
| rs2470404 | chr12 | 2049448 | 2049449 | No |
| rs2470425 | chr12 | 2077896 | 2077897 | No |
| rs2534715 | chr12 | 6573145 | 6573146 | No |
| rs2534717 | chr12 | 6573748 | 6573749 | No |
| rs2534721 | chr12 | 6580143 | 6580144 | No |
| rs2607916 | chr12 | 716508 | 716509 | No |
| rs2887533 | chr12 | 1201545 | 1201546 | No |
| rs2887571 | chr12 | 1638170 | 1638171 | No |
| rs2887780 | chr12 | 2499625 | 2499626 | Yes |
| rs2906892 | chr12 | 1995621 | 1995622 | No |
| rs2906893 | chr12 | 1995698 | 1995699 | No |
| rs2907494 | chr12 | 4642183 | 4642184 | No |
| rs2907495 | chr12 | 4638163 | 4638164 | No |
| rs2907499 | chr12 | 4616900 | 4616901 | No |
| rs2970808 | chr12 | 4640361 | 4640362 | No |
| rs2970821 | chr12 | 4519122 | 4519123 | No |
| rs3217830 | chr12 | 4392529 | 4392530 | No |
| rs3217840 | chr12 | 4394876 | 4394877 | No |
| rs3217896 | chr12 | 4404157 | 4404158 | No |
| rs3217898 | chr12 | 4404375 | 4404376 | No |
| rs3217933 | chr12 | 4412999 | 4413000 | No |
| rs35452680 | chr12 | 4277661 | 4277662 | No |
| rs367206 | chr12 | 5757028 | 5757029 | No |
| rs367978 | chr12 | 5756823 | 5756824 | No |
| rs369858 | chr12 | 5801347 | 5801348 | No |
| rs3739127 | chr2 | 131674284 | 131674285 | No |
| rs3741935 | chr12 | 3701389 | 3701390 | No |
| rs3759371 | chr12 | 464253 | 464254 | No |
| rs376708 | chr12 | 5762979 | 5762980 | No |
| rs3782586 | chr12 | 5690884 | 5690885 | No |
| rs3782614 | chr12 | 5775102 | 5775103 | No |
| rs3782642 | chr12 | 5850146 | 5850147 | No |
| rs3782645 | chr12 | 5888252 | 5888253 | No |
| rs3782751 | chr12 | 3673498 | 3673499 | No |
| rs3782752 | chr12 | 3670150 | 3670151 | No |
| rs3782753 | chr12 | 3670038 | 3670039 | No |
| rs3782761 | chr12 | 3652625 | 3652626 | No |
| rs3782803 | chr12 | 3348436 | 3348437 | No |
| rs3782805 | chr12 | 3345978 | 3345979 | No |
| rs3782843 | chr12 | 3113579 | 3113580 | No |
| rs3782860 | chr12 | 361995 | 361996 | No |
| rs3794297 | chr12 | 2338395 | 2338396 | Yes |
| rs380709 | chr12 | 5762089 | 5762090 | No |
| rs3809257 | chr12 | 3864403 | 3864404 | No |
| rs3809263 | chr12 | 773455 | 773456 | No |
| rs381373 | chr12 | 5755326 | 5755327 | No |
| rs3819526 | chr12 | 2436521 | 2436522 | Yes |
| rs3819532 | chr12 | 2436836 | 2436837 | Yes |
| rs3819535 | chr12 | 2436883 | 2436884 | Yes |
| rs3819537 | chr12 | 6193095 | 6193096 | No |
| rs3825369 | chr12 | 3109640 | 3109641 | No |
| rs3851333 | chr2 | 45545406 | 45545407 | No |
| rs3858698 | chr12 | 2115760 | 2115761 | No |
| rs3858703 | chr12 | 863516 | 863517 | No |
| rs387896 | chr12 | 5795450 | 5795451 | No |
| rs406430 | chr12 | 5753919 | 5753920 | No |
| rs4126711 | chr12 | 2227310 | 2227311 | Yes |
| rs4147666 | chr12 | 4757354 | 4757355 | No |
| rs4147667 | chr12 | 4757392 | 4757393 | No |
| rs4147672 | chr12 | 4764136 | 4764137 | No |
| rs417642 | chr12 | 5763771 | 5763772 | No |
| rs4238022 | chr12 | 5482395 | 5482396 | No |
| rs4238023 | chr12 | 5524561 | 5524562 | No |
| rs4283041 | chr12 | 1637912 | 1637913 | No |
| rs4298967 | chr12 | 2408193 | 2408194 | Yes |
| rs4370987 | chr12 | 2494925 | 2494926 | Yes |
| rs4417359 | chr12 | 6293321 | 6293322 | No |
| rs4437002 | chr2 | 131717890 | 131717891 | No |
| rs4441076 | chr12 | 2502057 | 2502058 | Yes |
| rs444834 | chr12 | 5756794 | 5756795 | No |
| rs445467 | chr12 | 5756322 | 5756323 | No |
| rs4477507 | chr12 | 4373927 | 4373928 | No |
| rs4530408 | chr2 | 131693108 | 131693109 | No |
| rs453385 | chr12 | 5759608 | 5759609 | No |
| rs454704 | chr12 | 5758661 | 5758662 | No |
| rs454736 | chr12 | 5758619 | 5758620 | No |
| rs4631963 | chr12 | 4966213 | 4966214 | No |
| rs4764487 | chr12 | 6332837 | 6332838 | No |
| rs4764519 | chr12 | 5937957 | 5937958 | No |
| rs4764589 | chr12 | 6532281 | 6532282 | No |
| rs4764605 | chr12 | 6623388 | 6623389 | No |
| rs4765670 | chr12 | 2313125 | 2313126 | Yes |
| rs4765676 | chr12 | 2489313 | 2489314 | Yes |
| rs4765680 | chr12 | 2557098 | 2557099 | Yes |
| rs4765743 | chr12 | 3727524 | 3727525 | No |
| rs4765746 | chr12 | 3729882 | 3729883 | No |
| rs4765747 | chr12 | 3735619 | 3735620 | No |
| rs4765804 | chr12 | 5190351 | 5190352 | No |
| rs4765829 | chr12 | 1720793 | 1720794 | No |
| rs4765830 | chr12 | 1721253 | 1721254 | No |
| rs4765870 | chr12 | 2090875 | 2090876 | No |
| rs4765902 | chr12 | 2312964 | 2312965 | Yes |
| rs4765904 | chr12 | 2332392 | 2332393 | Yes |
| rs4765905 | chr12 | 2349583 | 2349584 | Yes |
| rs4765913 | chr12 | 2419895 | 2419896 | Yes |
| rs4765914 | chr12 | 2420376 | 2420377 | Yes |
| rs4765960 | chr12 | 2667929 | 2667930 | Yes |
| rs4766046 | chr12 | 3222713 | 3222714 | No |
| rs4766096 | chr12 | 3403998 | 3403999 | No |
| rs4766108 | chr12 | 3460878 | 3460879 | No |
| rs4766120 | chr12 | 3555525 | 3555526 | No |
| rs4766156 | chr12 | 3761921 | 3761922 | No |
| rs4766255 | chr12 | 4684230 | 4684231 | No |
| rs4766257 | chr12 | 4689012 | 4689013 | No |
| rs4766312 | chr12 | 5031589 | 5031590 | No |
| rs4766335 | chr12 | 5168848 | 5168849 | No |
| rs482514 | chr12 | 396966 | 396967 | No |
| rs492955 | chr12 | 5241914 | 5241915 | No |
| rs4930739 | chr12 | 5712477 | 5712478 | No |
| rs4930764 | chr12 | 5702444 | 5702445 | No |
| rs4930766 | chr12 | 5712163 | 5712164 | No |
| rs4980804 | chr12 | 261928 | 261929 | No |
| rs4980833 | chr12 | 632239 | 632240 | No |
| rs4980836 | chr12 | 675440 | 675441 | No |
| rs4980877 | chr12 | 418915 | 418916 | No |
| rs4980885 | chr12 | 442619 | 442620 | No |
| rs4980968 | chr12 | 883177 | 883178 | No |
| rs4980976 | chr12 | 232703 | 232704 | No |
| rs524468 | chr12 | 371785 | 371786 | No |
| rs529446 | chr12 | 5221318 | 5221319 | No |
| rs550011 | chr12 | 5240225 | 5240226 | No |
| rs555044 | chr12 | 335921 | 335922 | No |
| rs564292 | chr12 | 5235861 | 5235862 | No |
| rs576159 | chr12 | 382875 | 382876 | No |
| rs579526 | chr12 | 373119 | 373120 | No |
| rs611652 | chr12 | 5239768 | 5239769 | No |
| rs614616 | chr12 | 3365072 | 3365073 | No |
| rs622513 | chr12 | 4071237 | 4071238 | No |
| rs6430431 | chr2 | 131684662 | 131684663 | No |
| rs6430441 | chr2 | 131695641 | 131695642 | No |
| rs6489338 | chr12 | 2060579 | 2060580 | No |
| rs6489341 | chr12 | 2104467 | 2104468 | No |
| rs6489353 | chr12 | 2338857 | 2338858 | Yes |
| rs6489367 | chr12 | 2475675 | 2475676 | Yes |
| rs6489375 | chr12 | 2777767 | 2777768 | Yes |
| rs6489439 | chr12 | 3227202 | 3227203 | No |
| rs6489454 | chr12 | 475793 | 475794 | No |
| rs6489482 | chr12 | 3709764 | 3709765 | No |
| rs6489500 | chr12 | 3993627 | 3993628 | No |
| rs6489501 | chr12 | 3993808 | 3993809 | No |
| rs6489568 | chr12 | 4930099 | 4930100 | No |
| rs6489589 | chr12 | 5100436 | 5100437 | No |
| rs6489630 | chr12 | 5604623 | 5604624 | No |
| rs6489662 | chr12 | 726014 | 726015 | No |
| rs6489746 | chr12 | 865969 | 865970 | No |
| rs6547780 | chr2 | 88811786 | 88811787 | No |
| rs6547781 | chr2 | 88831690 | 88831691 | No |
| rs6718310 | chr2 | 131688178 | 131688179 | No |
| rs6718816 | chr2 | 131688586 | 131688587 | No |
| rs7131705 | chr12 | 2186579 | 2186580 | Yes |
| rs7132154 | chr12 | 2461222 | 2461223 | Yes |
| rs7132763 | chr12 | 487334 | 487335 | No |
| rs7133350 | chr12 | 3743474 | 3743475 | No |
| rs7134353 | chr12 | 425501 | 425502 | No |
| rs7134570 | chr12 | 1202357 | 1202358 | No |
| rs7135976 | chr12 | 6186115 | 6186116 | No |
| rs7137113 | chr12 | 3226697 | 3226698 | No |
| rs7137875 | chr12 | 3697838 | 3697839 | No |
| rs7138047 | chr12 | 5418537 | 5418538 | No |
| rs714775 | chr12 | 6619401 | 6619402 | No |
| rs715230 | chr12 | 461597 | 461598 | No |
| rs717596 | chr12 | 3841342 | 3841343 | No |
| rs720721 | chr12 | 2667743 | 2667744 | Yes |
| rs721159 | chr12 | 1948976 | 1948977 | No |
| rs724709 | chr12 | 898399 | 898400 | No |
| rs7294326 | chr12 | 594173 | 594174 | No |
| rs7295590 | chr12 | 2442788 | 2442789 | Yes |
| rs7295704 | chr12 | 884888 | 884889 | No |
| rs7296925 | chr12 | 3715460 | 3715461 | No |
| rs7297582 | chr12 | 2355805 | 2355806 | Yes |
| rs7297784 | chr12 | 5143053 | 5143054 | No |
| rs7297853 | chr12 | 3751045 | 3751046 | No |
| rs7298053 | chr12 | 6619053 | 6619054 | No |
| rs7298357 | chr12 | 6000125 | 6000126 | No |
| rs7298570 | chr12 | 6581481 | 6581482 | No |
| rs7298766 | chr12 | 661655 | 661656 | No |
| rs7300043 | chr12 | 3958712 | 3958713 | No |
| rs7300338 | chr12 | 4139899 | 4139900 | No |
| rs7300444 | chr12 | 993929 | 993930 | No |
| rs7301134 | chr12 | 3508718 | 3508719 | No |
| rs7301422 | chr12 | 447750 | 447751 | No |
| rs7302743 | chr12 | 2269587 | 2269588 | Yes |
| rs7303991 | chr12 | 1956289 | 1956290 | No |
| rs7304949 | chr12 | 4609624 | 4609625 | No |
| rs7304958 | chr12 | 3225938 | 3225939 | No |
| rs7305265 | chr12 | 2032112 | 2032113 | No |
| rs7305676 | chr12 | 5706126 | 5706127 | No |
| rs7307214 | chr12 | 3851048 | 3851049 | No |
| rs7308641 | chr12 | 4667909 | 4667910 | No |
| rs7309847 | chr12 | 3841651 | 3841652 | No |
| rs7312402 | chr12 | 4755516 | 4755517 | No |
| rs7314661 | chr12 | 779905 | 779906 | No |
| rs7314691 | chr12 | 661980 | 661981 | No |
| rs7315460 | chr12 | 5235996 | 5235997 | No |
| rs7315852 | chr12 | 417632 | 417633 | No |
| rs7316135 | chr12 | 1718073 | 1718074 | No |
| rs7342306 | chr12 | 6291092 | 6291093 | No |
| rs735295 | chr12 | 547682 | 547683 | No |
| rs740355 | chr12 | 1723227 | 1723228 | No |
| rs740416 | chr12 | 2499891 | 2499892 | Yes |
| rs740417 | chr12 | 2499848 | 2499849 | Yes |
| rs740458 | chr12 | 1989413 | 1989414 | No |
| rs740897 | chr12 | 5441266 | 5441267 | No |
| rs740898 | chr12 | 5441311 | 5441312 | No |
| rs740901 | chr12 | 5444139 | 5444140 | No |
| rs7485896 | chr12 | 5938445 | 5938446 | No |
| rs7486388 | chr12 | 5766337 | 5766338 | No |
| rs7488279 | chr12 | 5568219 | 5568220 | No |
| rs7488316 | chr12 | 5606371 | 5606372 | No |
| rs753076 | chr12 | 2499167 | 2499168 | Yes |
| rs7579148 | chr2 | 131706882 | 131706883 | No |
| rs758162 | chr12 | 1966288 | 1966289 | No |
| rs758170 | chr12 | 2361459 | 2361460 | Yes |
| rs758173 | chr12 | 2315227 | 2315228 | Yes |
| rs758174 | chr12 | 2315705 | 2315706 | Yes |
| rs758563 | chr12 | 2737058 | 2737059 | Yes |
| rs758739 | chr12 | 6626368 | 6626369 | No |
| rs758798 | chr12 | 5441540 | 5441541 | No |
| rs7595875 | chr2 | 88817091 | 88817092 | No |
| rs763385 | chr12 | 2087044 | 2087045 | No |
| rs763580 | chr12 | 6193536 | 6193537 | No |
| rs7645622 | chr3 | 145887758 | 145887759 | No |
| rs764614 | chr12 | 430651 | 430652 | No |
| rs765124 | chr12 | 2156144 | 2156145 | No |
| rs765125 | chr12 | 2156206 | 2156207 | No |
| rs765891 | chr12 | 966992 | 966993 | No |
| rs769087 | chr12 | 2344643 | 2344644 | Yes |
| rs7953137 | chr12 | 486832 | 486833 | No |
| rs7954351 | chr12 | 6163704 | 6163705 | No |
| rs7954694 | chr12 | 3989666 | 3989667 | No |
| rs7955352 | chr12 | 4946014 | 4946015 | No |
| rs7957013 | chr12 | 3425576 | 3425577 | No |
| rs7957545 | chr12 | 2324041 | 2324042 | Yes |
| rs7958182 | chr12 | 4768436 | 4768437 | No |
| rs7961089 | chr12 | 2463053 | 2463054 | Yes |
| rs7961557 | chr12 | 2493590 | 2493591 | Yes |
| rs7961826 | chr12 | 4973358 | 4973359 | No |
| rs7963651 | chr12 | 4297830 | 4297831 | No |
| rs7965755 | chr12 | 4669739 | 4669740 | No |
| rs7966355 | chr12 | 2075651 | 2075652 | No |
| rs7967032 | chr12 | 2046098 | 2046099 | No |
| rs7968633 | chr12 | 2734449 | 2734450 | Yes |
| rs7968680 | chr12 | 2500202 | 2500203 | Yes |
| rs7968928 | chr12 | 5411035 | 5411036 | No |
| rs7969171 | chr12 | 1679783 | 1679784 | No |
| rs7969761 | chr12 | 355841 | 355842 | No |
| rs7970204 | chr12 | 3429755 | 3429756 | No |
| rs7972490 | chr12 | 1009055 | 1009056 | No |
| rs7972545 | chr12 | 3840771 | 3840772 | No |
| rs7972667 | chr12 | 863832 | 863833 | No |
| rs7972920 | chr12 | 4768618 | 4768619 | No |
| rs7975360 | chr12 | 6605154 | 6605155 | No |
| rs7976678 | chr12 | 6537543 | 6537544 | No |
| rs7976964 | chr12 | 887925 | 887926 | No |
| rs7977039 | chr12 | 2615133 | 2615134 | Yes |
| rs797765 | chr12 | 372437 | 372438 | No |
| rs7978349 | chr12 | 3694487 | 3694488 | No |
| rs7978411 | chr12 | 5415849 | 5415850 | No |
| rs7978603 | chr12 | 6598072 | 6598073 | No |
| rs7979389 | chr12 | 2441461 | 2441462 | Yes |
| rs7980163 | chr12 | 955507 | 955508 | No |
| rs8181786 | chr12 | 2019321 | 2019322 | No |
| rs82602 | chr12 | 2760002 | 2760003 | Yes |
| rs867167 | chr12 | 5259442 | 5259443 | No |
| rs876503 | chr12 | 2797178 | 2797179 | Yes |
| rs877478 | chr12 | 229946 | 229947 | No |
| rs880342 | chr12 | 2481606 | 2481607 | Yes |
| rs882194 | chr12 | 2350451 | 2350452 | Yes |
| rs882195 | chr12 | 2350400 | 2350401 | Yes |
| rs886540 | chr12 | 1590757 | 1590758 | No |
| rs886541 | chr12 | 1590898 | 1590899 | No |
| rs886898 | chr12 | 2481935 | 2481936 | Yes |
| rs886941 | chr12 | 2026914 | 2026915 | No |
| rs887357 | chr12 | 3474644 | 3474645 | No |
| rs917367 | chr12 | 1967525 | 1967526 | No |
| rs917668 | chr12 | 5452772 | 5452773 | No |
| rs936577 | chr12 | 5263803 | 5263804 | No |
| rs959940 | chr3 | 65148430 | 65148431 | No |
| rs959941 | chr3 | 65148831 | 65148832 | No |
| rs9651889 | chr12 | 257059 | 257060 | No |
| rs9669580 | chr12 | 5811468 | 5811469 | No |
| rs979878 | chr12 | 4757473 | 4757474 | No |

SNP IDs listed in the above table correspond to the Reference SNP (refSNP) Cluster Report No. of the NCBI dbSNP database located at http://www.ncbi.nlm.nih.gov/snp. dbSNP is a database of single nucleotide polymorphisms (SNPs) and multiple small-scale variations

In exemplary aspects, the method further comprises administering a therapeutic compound other than a CCB, such as a mood stabilizer. As used herein, the term “mood stabilizer” refers to a psychiatric medication used to treat mood disorders. The mood stabilizer may be include, but are not limited to, Lithium, Valproic acid, Lamotrigine, Carbamazepine, Divalproex sodium, Sodium valproate, Lithium carbonate, Lithium hydroxidelithium. In exemplary aspects, the mood disorder is an anticonvulsant or an atypical antipsychotic.

In exemplary aspects, the method comprises sample preparation steps. DNA can be extracted from whole blood or saliva sample obtained from the subject. Genotyping can be performed on the DNA using PsychChip or TaqMan method, or other methods known in the art, to determine the subject's genotype, including for the allele [A] of CACNA1C, wherein allele [A] comprises the sequence of polymorphic marker rs1006737.

For example, in some aspects, the method comprises selecting a specific cell population from the sample obtained from the subject. The selection step may be carried out by any means known in the art, including, but not limited to FACS or chromatography. In order to assess gene expression level, e.g., RNA levels or protein levels, in neuronal cells, fibroblast cells can be obtained from the subject and used to construct induced pluripotent stem cells (iPSCs). Then, iPSCs can be induced into neuronal cells in culture. Methods for obtaining iPSCs and inducing them to become neuronal cells in culture are known in the art. Gene expression of CACNA1C can be assessed in the cultured neuronal cells using methods of detecting RNA or protein that are known and used routinely in the art and described herein. In exemplary aspects, wherein RNA expression levels are measured, the method may further comprise a step to extract or isolate the RNA from the cells of the sample.

Any and all possible combinations of the steps described herein are contemplated for purposes of the inventive methods

Mood Disorders

As used herein, the term “mood disorder” refers to a mental disorder wherein a disturbance in a subject's mood is thought to be the main underlying feature. Mood disorders are classified in the Diagnostic and Statistical Manual(s) of Mental Disorders, DSM-IV and DSM5. In exemplary embodiments, the mood disorder is a depressive disorder, including, but not limited to, atypical depression, melancholic depression, psychotic major depression, catatonic depression, postpartum depression, seasonal affective disorder, dysthymia, double depression, depressive disorder not otherwise specified, depressive personality disorder, recurrent brief depression, and mino depressive disorder. In exemplary embodiments, the mood disorder is a bipolar disorder (BD). In exemplary aspects, the bipolar disorder is mania, hypomania, bipolar I, bipolar II, cyclothymia, or biopolar disorder not otherwise specified.

Samples

With regard to the methods disclosed herein, in exemplary embodiments, the sample obtained from the subject comprises a bodily fluid, including, but not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of the foregoing samples. In exemplary aspects, the sample comprises DNA obtained from one or more blood cells of the subject. In exemplary aspects, the sample comprises DNA obtained from saliva of the subject. In exemplary aspects, the sample comprises brain or neuronal cells from the subject. In exemplary aspects, the sample contains neuronal cells derived from iPSCs induced from fibroblasts collected from the subject. In exemplary aspects, the sample comprises RNA from neuronal cells derived from such iPSCs.

Subjects

With regard to the methods disclosed herein, the subject in exemplary aspects is a mammal, preferably a human. In exemplary aspects, the subject is an adult. In exemplary aspects, the subject is a female. In exemplary aspects, the subject is a male. In exemplary aspects, the subject is a subject with a mood disorder. In exemplary aspects, the mood disorder is any of those mentioned herein. In exemplary aspects, the subject is one who suffers from a bipolar disorder. In exemplary aspects, the subject has been treated with a CCB. In exemplary aspects, the subject has not been treated with a CCB.

Calcium Channel Blockers

As used herein, the term “Calcium Channel Blocker” or CCB″ refers to any compound, e.g., any drug, that blocks the action of a calcium channel. In exemplary embodiments, the CCB is selected from the group consisting of: amlodipine, diltiazem, felodipine, isradipine, nicardipine, nifedipine, nisoldipine and verapamil. In exemplary aspects, the calcium channel blocker is nicardipine.

Formulations and Routes of Administration

With regard to the administration of a therapeutic agent, e.g., a CCB, the agent may be administered through any suitable means, compositions and routes known in the art.

Kits

The invention further provides kits which in exemplary embodiments are useful in the methods described herein. In exemplary embodiments, the kit comprises one or more binding agents to the CACNA1C gene or a gene product thereof. In exemplary aspects, the binding agent is a nucleic acid molecule which is about 5, about 10, about 15, about 20, about 25, about 30, about 35, about 40, about 45 or about 50 nucleotides in length. In exemplary aspects, the nucleic acid molecule is about 15 to about 30 nucleotides in length or about 20 to 30 nucleotides in length or about 25 to 30 nucleotides in length. In exemplary aspects, the nucleic acid molecule is about 25 nucleotides in length.

In exemplary aspects, the binding agent, e.g., the nucleic acid molecule, is conjugated to a detectable label. The detectable label, in exemplary aspects, is a radioisotope, a fluorophore, or an element particle.

In exemplary embodiments, the binding agent is a nucleic acid molecule, e.g., a nucleic acid probe, which specifically binds to the CACNA1C gene or a gene product thereof. In exemplary embodiments, the nucleic acid molecule hybridizes to or specifically binds to SEQ ID NO: 1, and in exemplary aspects, the nucleic acid molecule hybridizes to or specifically binds to a region of SEQ ID NO: 1 which is about 20 basepairs (bp) to about 1000 bp (e.g., about 30 bp, about 40 bp, about 50 bp, about 60 bp, about 70 bp, about 80 bp, about 90 bp, about 100 bp, about 200 bp, about 300 bp, about 400 bp, about 500 bp, about 600 bp, about 700 bp, about 800 bp, about 900 bp, about 1000 bp) upstream or downstream of position 270344 of SEQ ID NO: 1, which is the polymorphic site of rs1006737. In exemplary aspects, the nucleic acid molecule hybridizes to or specifically binds to a region of SEQ ID NO: 1 which is about 50 bp to about 500 bp upstream or downstream of position 270344 of SEQ ID NO: 1. In exemplary aspects, the nucleic acid molecule hybridizes to or specifically binds to a region of SEQ ID NO: 1 which is about 50 bp to about 400 bp upstream or downstream of position 270344 of SEQ ID NO: 1. In exemplary aspects, the nucleic acid molecule hybridizes to or specifically binds to a region of SEQ ID NO: 1 which is about 50 bp to about 300 bp upstream or downstream of position 270344 of SEQ ID NO: 1. In exemplary aspects, the nucleic acid molecule hybridizes to or specifically binds to a region of SEQ ID NO: 1 which is about 50 bp to about 200 bp upstream or downstream of position 270344 of SEQ ID NO: 1. In exemplary aspects, the nucleic acid molecule hybridizes to or specifically binds to a region of SEQ ID NO: 1 which is about 50 bp to about 100 bp upstream or downstream of position 270344 of SEQ ID NO: 1. In exemplary aspects, the nucleic acid molecule hybridizes to or specifically binds to a region of SEQ ID NO: 1 which is about 100 bp to about 500 bp upstream or downstream of position 270344 of SEQ ID NO: 1. In exemplary aspects, the nucleic acid molecule hybridizes to or specifically binds to a region of SEQ ID NO: 1 which is about 200 bp to about 500 bp upstream or downstream of position 270344 of SEQ ID NO: 1. In exemplary aspects, the nucleic acid molecule hybridizes to or specifically binds to a region of SEQ ID NO: 1 which is about 300 bp to about 500 bp upstream or downstream of position 270344 of SEQ ID NO: 1. In exemplary aspects, the nucleic acid molecule hybridizes to or specifically binds to a region of SEQ ID NO: 1 which is about 400 bp to about 500 bp upstream or downstream of position 270344 of SEQ ID NO: 1.

In exemplary aspects, the kit comprises a pair of primers suitable for, e.g., amplification of a region of the CACNA1C gene of a given sample. In exemplary aspects, the pair comprises two nucleic acid molecules which work together to amplify (e.g., via a polymerase chain reaction (PCR)) a region of the CACNA1C gene. In exemplary aspects, at least one primer of the pair is a nucleic acid described above.

In exemplary aspects, the binding agent is a set of oligonucleotides that specifically bind to the CACNA1C gene, and flank the sequence comprising position 2282 of SEQ ID NO: 2. In exemplary aspects, the kit comprises a primer pair, wherein the forward primer is CCACTTGGCTCTATCAAAGTCT and the reverse primer is CCTGAGAGACACTGTGAGGT (SEQ ID NO: 50 and 51, respectively). In exemplary aspects, the kit comprises the primer pairs attached to a solid support. In exemplary aspects, the solid support is a multi-well plate and the primer pairs are attached to a well of the multi-well plate.

In exemplary aspects, the nucleic acid molecule, primer and/or probe of the kit comprises one or more non-naturally-occurring nucleotides and/or non-naturally-occurring internucleotide linkages (e.g., phosphoroamidate linkages, phosphorothioate linkages). In exemplary aspects, the nucleic acid molecule, primer and/or probe of the kit comprises at least one non-naturally-occurring nucleotide and/or non-naturally-occurring internucleotide linkage. In exemplary aspects, the nucleic acid molecule, primer and/or probe of the kit comprises one or more modified nucleotides, including, but not limited to, 5-fluorouracil, 5-bromouracil, 5-chlorouracil, 5-iodouracil, hypoxanthine, xanthine, 4-acetylcytosine, 5-(carboxyhydroxymethyl)uracil, 5-carboxymethylaminomethyl-2-thiouridine, 5-carboxymethylaminomethyluracil, dihydrouracil, beta-D-galactosylqueuosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N -substituted adenine, 7-methylguanine, 5-methylammomethyluracil, 5-methoxyaminomethyl-2-thiouracil, beta-D-mannosylqueuosine, 5′-methoxycarboxymethyluracil, 5-methoxyuracil, 2-methylthio-N⁶-isopentenyladenine, uracil-5-oxyacetic acid (v), wybutoxosine, pseudouracil, queuosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl)uracil, and 2,6-diaminopurine.

In exemplary aspects, the nucleic acid molecule, primer and/or probe of the kit comprises non-naturally-occurring nucleotides which differ from naturally occurring nucleotides by comprising a chemical group in place of the phosphate group. In exemplary aspects, the nucleic acid molecule, primer and/or probe of the kit comprises or is a methylphosphonate oligonucleotide, which are noncharged oligomers in which a non-bridging oxygen atom, e.g., alpha oxygen of the phosphate, is replaced by a methyl group. In exemplary aspects, the nucleic acid molecule, primer and/or probe of the kit comprises or is a phosphorothioate, wherein at least one of the non-bridging oxygen atom, e.g., alpha oxygen of the phosphate, is replaced by a sulfur. In exemplary aspects, the nucleic acid molecule, primer and/or probe of the kit comprises or is a boranophosphate olignucleotide, wherein at least one of the non-bridging oxygen atom, e.g., alpha oxygen of the phosphate, is replaced by —BH₃.

In exemplary aspects, the nucleic acid molecule, primer and/or probe of the kit comprises at least one non-naturally-occurring nucleotide which differs from naturally occurring nucleotides by comprising a ring structure other than ribose or 2-deoxyribose. In exemplary aspects, the nucleic acid molecule, primer and/or probe of the kit is an analog comprising a replacement of the hydroxyl at the 2′-position of ribose with an O-alkyl group, e.g., —O—CH₃, —OCH₂CH₃. In exemplary aspects, the nucleic acid molecule, primer and/or probe of the kit comprises a modified ribonucleotide wherein the 2′ hydroxyl of ribose is modified to methoxy (OMe) or methoxy-ethyl (MOE) group. In exemplary aspects, the nucleic acid molecule, primer and/or probe of the kit comprises a modified ribonucleotide wherein the 2′ hydroxyl of ribose is replaced with allyl, amino, azido, halo, thio, O-allyl, O—C₁-C₁₀ alkyl, O—C₁-C₁₀ substituted alkyl, O—C₁-C₁₀ alkoxy, O—C₁-C₁₀ substituted alkoxy, OCF₃, O(CH₂)₂SCH₃, O(CH₂)₂—O—N(R¹)(R²), or O(CH₂)—C(═O)—N(R¹)(R²), wherein each of R¹ and R² is independently selected from the group consisting of H, an amino protecting group or substituted or unsubstituted C₁-C₁₀ alkyl. In exemplary aspects, the antisense nucleic acid analog comprises a modified ribonucleotide wherein the 2′ hydroxyl of ribose is replaced with 2′F, SH, CN, OCN, CF₃, O-alkyl, S-Alkyl, N(R¹)alkyl, O-alkenyl, S-alkenyl, or N(R¹)-alkenyl, O-alkynyl, S-alkynyl, N(R¹)-alkynyl, O-alkylenyl, O-Alkyl, alknyyl, alkaryl, aralkyl, O-alkaryl, or O-aralkyl. In exemplary aspects, the nucleic acid molecule, primer and/or probe of the kit is an analog comprising a replacement of the hydrogen at the 2′-position of ribose with halo, e.g., F. In exemplary aspects, the antisense nucleic acid analog comprises a fluorine derivative nucleic acid.

In exemplary aspects, the nucleic acid molecule, primer and/or probe of the kit comprises a substituted ring. In exemplary aspects, the nucleic acid molecule, primer and/or probe of the kit is or comprises a hexitol nucleic acid. In exemplary aspects, the nucleic acid molecule, primer and/or probe of the kit is or comprises a nucleotide with a bicyclic or tricyclic sugar moiety. In exemplary aspects, the bicyclic sugar moiety comprises a bridge between the 4′ and 2′ furanose ring atoms. Examplary moieties include, but are not limited to: —[C(Rₐ)(R_{b})]ₙ—, —[C(Rₐ)(R_{b})]ₙ-0-, —C(RₐR_{b})-N(R)-0- or, —C(RₐR_{b})-0-N(R)—; 4′-CH₂-2′,4′-(CH₂)₂-2′,4′- (CH₂)₃-2′,4′-(CH₂)-0-2′ (LNA); 4′-(CH₂)—S-2′; 4′-(CH₂)₂-0-2′ (ENA); 4′-CH(CH₃)-0-2′ (cEt) and 4′- CH(CH₂OCH₃)-0-2′,4′-C(CH₃)(CH₃)-0-2′,4′-CH₂—N(OCH₃)-2′,4′-CH₂-0-N(CH₃)-2′4′-CH₂-0-N(R)-2′, and 4′-CH₂—N(R)-0-2′-, wherein each R is, independently, H, a protecting group, or C₁C₁₂ alkyl; 4′-CH₂—N(R)-0-2′, wherein R is H, C1-C12 alkyl, or a protecting group, 4′-CH₂—C(H)(CH₃)-2′,4′-CH₂—C(═CH₂)-2′. Such molecules are known in the art. See, e.g., International Application Publication No. WO 2008/154401, U.S. Pat. No. 7,399,845, International Application Publication No. WO2009/006478, International Application Publication No. WO2008/150729, U.S. Application Publication No. US2004/0171570, U.S. Pat. No. 7,427,672, and Chattopadhyaya, et al, J. Org. Chem., 2009, 74, 118-134). In exemplary aspects, the the nucleic acid molecule, primer and/or probe of the kit comprises a nucleoside comprising a bicyclic sugar moiety, or a bicyclic nucleoside (BNA). In exemplary aspects, the nucleic acid molecule, primer and/or probe of the kit comprises a BNA selected from the group consisting of: α-L-Methyleneoxy (4′-CH₂-0-2′) BNA, Aminooxy (4′-CH₂-0-N(R)-2′) BNA, β-D-Methyleneoxy (4′-CH₂-0-2′) BNA, Ethyleneoxy (4′-(CH₂)₂-0-2′) BNA, methylene-amino (4′-CH2-N(R)-2′) BNA, methyl carbocyclic (4′-CH₂—CH(CH₃)-2′) BNA, Methyl(methyleneoxy) (4′-CH(CH₃)-0-2′) BNA (also known as constrained ethyl or cEt), methylene-thio (4′-CH₂-S-2′) BNA, Oxyamino (4′-CH₂—N(R)-0-2′) BNA, and propylene carbocyclic (4′-(CH₂)₃-2′) BNA. Such BNAs are described in the art. See, e.g., International Patent Publication No. WO 2014/071078.

In exemplary aspects, the nucleic acid molecule, primer and/or probe of the kit comprises a modified backbone. In exemplary aspects, the nucleic acid molecule, primer and/or probe of the kit is or comprises a peptide nucleic acid (PNA) containing an uncharged flexible polyamide backbone comprising repeating N-(2-aminoethyl)glycine units to which the nucleobases are attached via methylene carbonyl linkers. In exemplary aspects, the nucleic acid molecule, primer and/or probe of the kit comprises a backbone substitution. In exemplary aspects, the nucleic acid molecule, primer and/or probe of the kit is or comprises an N3′→P5′ phosphoramidate, which results from the replacement of the oxygen at the 3′ position on ribose by an amine group. Such nucleic acid analogs are further described in Dias and Stein, Molec Cancer Ther 1: 347-355 (2002). In exemplary aspects, the nucleic acid molecule, primer and/or probe of the kit comprises a nucleotide comprising a conformational lock. In exemplary aspects, the nucleic acid molecule, primer and/or probe of the kit is or comprises a locked nucleic acid.

In exemplary aspects, the nucleic acid molecule, primer and/or probe of the kit comprises a 6-membered morpholine ring, in place of the ribose or 2-deoxyribose ring found in RNA or DNA. In exemplary aspects, the nucleic acid molecule, primer and/or probe of the kit comprises non-ionic phophorodiamidate intersubunit linkages in place of anionic phophodiester linkages found in RNA and DNA. In exemplary aspects, the nucleic acid molecule, primer and/or probe of the kit comprises nucleobases (e.g., adenine (A), cytosine (C), guanine (G), thymine, thymine (T), uracil (U)) found in RNA and DNA. In exemplary aspects, the nucleic acid molecule, primer and/or probe of the kit is a Morpholino oligomer comprising a polymer of subunits, each subunit of which comprises a 6-membered morpholine ring and a nucleobase (e.g., A, C, G, T, U), wherein the units are linked via non-ionic phophorodiamidate intersubunit linkages. For purposes herein, when referring to the sequence of a Morpholino oligomer, the conventional single-letter nucleobase codes (e.g., A, C, G, T, U) are used to refer to the nucleobase attached to the morpholine ring.

In exemplary aspects, the kit comprises reagents for measuring the expression level of the CACNA1C gene. For example, antibodies that bind to the CACNA1C protein CAᵥ1.2 may be included in the kit of the invention. The antibody may be any type of immunoglobulin, fragment of an immunoglobulin, or nonantibody scaffold known in the art. In exemplary embodiments, the antibody is an antibody of isotype IgA, IgD, IgE, IgG, or IgM. Also, the antibody in some embodiments is a monoclonal antibody. In other embodiments, the antibody is a polyclonal antibody. In some embodiments, the antibody is a naturally-occurring antibody, e.g., an antibody isolated and/or purified from a mammal, or produced by a hybridoma generated from a mammalian cell. Methods of producing antibodies are well known in the art. In some embodiments, the antibody is a genetically-engineered antibody, e.g., a single chain antibody, a humanized antibody, a chimeric antibody, a CDR-grafted antibody, a humaneered antibody, a bispecific antibody, a trispecific antibody, a recombinant antibody, and the like. Genetic engineering techniques also provide the ability to make fully human antibodies in a non-human source. In some aspects, the antibody is in polymeric, oligomeric, or multimeric form. In certain embodiments in which the antibody comprises two or more distinct antigen binding regions fragments, the antibody is considered bispecific, trispecific, or multi-specific, or bivalent, trivalent, or multivalent, depending on the number of distinct epitopes that are recognized and bound by the antibody.

In some aspects of the invention, the binding agent is an antigen binding fragment of an antibody. The antigen binding fragment (also referred to herein as “antigen binding portion”) may be an antigen binding fragment of any of the antibodies described herein. The antigen binding fragment can be any part of an antibody that has at least one antigen binding site, including, but not limited to, Fab, F(ab′)₂, dsFv, sFv, scFvs, diabodies, triabodies, bis-scFvs, fragments expressed by a Fab expression library, domain antibodies, VhH domains, V-NAR domains, VH domains, VL domains, and the like.

In exemplary aspects, the kit comprises a CCB, including but not limited to any of the CCBs described herein. In exemplary aspects, the kit comprises a container suitable for holding a sample obtained from the subject. In exemplary aspects, the kit comprises a vial, a tube, a microtiter plate, a dish, a flask, or the like. In exemplary aspects, the kit comprises reagents suitable for isolating DNA, RNA or proteins from the sample.

In exemplary aspects, the nonantibody scaffold is a nanobody, affibody, affilin, anticalin (lipocalin), fynomer, Kunitz variant, fibronectin type III (FN3) domain (monobody, and related binding protein systems using the FN3 domain), ankyrin repeat (DARPin), disulfide-constrained peptide, or other nonantibody scaffolds.

Computer Related Inventions

Related systems, e.g., computer systems, comprising a processor; a memory device coupled to the processor, and machine readable instructions stored on the memory device, are provided herein. In exemplary embodiments, the system comprises machine readable instructions that, when executed by the processor, cause the processor to: (a) receive a data value, α, that is a copy number determination of allele [A] of CACNA1C, wherein allele [A] comprises the sequence of polymorphic marker rs1006737, in the cells of a sample obtained from a subject; and (b) display an output relating to treating the subject for a mood disorder with a calcium channel blocker (CCB), when a is less than the diploid copy number of 2.

Related computer-readable storage media are also provided herein. In exemplary aspects, a computer-readable storage medium having stored thereon machine-readable instructions executable by a processor is provided. In exemplary aspects, the machine-readable instructions comprise: (a) instructions for receiving a data value, a, relating to the copy number of allele [A] of CACNA1C , wherein allele [A] comprises the sequence of the polymorphic marker rs1006737, in the cells of a sample obtained from a subject; and (b) instructions for displaying an output relating to treating the subject for a mood disorder with a calcium channel blocker (CCB), when a is less than the diploid copy number of 2.

Related methods implemented by a processor in a computer are provided herein. In exemplary embodiments, the method comprises (a) receiving a data value, α, relating to the copy number of allele [A] of CACNA1C, wherein allele [A] comprises the sequence of the polymorphic marker rs1006737, in the cells of a sample obtained from a subject; and (b) displaying an output relating to treating the subject for a mood disorder with a calcium channel blocker (CCB), when α is less than the diploid copy number of 2.

The following examples further illustrate the disclosure but, of course, should not be construed as in any way limiting its scope.

### EXAMPLES

### Example 1

This example demonstrates that an intronic SNP within the CACNA1C gene is a cis-expression quantitative trait locus for CACNA1C. This example also demonstrates that the risk allele is associated with decreased expression of this gene.

Abstract

Timothy Syndrome (TS) is caused by very rare exonic mutations of the CACNA1C gene that produce delayed inactivation of Cav1.2voltage-gated calcium channels during cellular action potentials, with greatly increased influx of calcium into the activated cells. The major clinical feature of this syndrome is a long QT interval that results in cardiac arrhythmias. However, TS also includes cognitive impairment, autism, and major developmental delays in many of the patients. We observed the appearance of Bipolar Disorder (BD) in a patient with a previously reported case of TS, who is one of the very few patients to survive childhood. This is most interesting because the common SNP most highly associated with BD is rs1006737, which we show here is a cis-expression quantitative trait locus (eQTL) for CACNA1C in human cerebellum, and the risk allele (A) is associated with decreased expression. To combine the CACNA1C perturbations in the presence of BD in this patient and in patients with the common CACNA1C SNP risk allele, we would propose that either increase or decrease in calcium influx in excitable cells can be associated with BD. In treatment of BD with calcium channel blocking drugs (CCBs), we would predict better response in patients without the risk allele, because they have increased CACNA1C expression.

Case Summary

The patient is a European-American male with TS Type 2 (see below) diagnosed in childhood. At age 28, the patient was referred for research evaluation after diagnosis of BD by one of us (FO). This was confirmed by interview with the patient and his mother, using the Diagnostic Interview for Genetic Studies (DIGS), version 3.0.

The patient was born full-term in good health, and had an uneventful history during his first three years. At age 4, he suffered a cardiac arrest during a trampoline class^{1,5}. Diagnostic work-up identified long QT syndrome, with a QT interval of 550-600 ms. In molecular investigation of his case and one other, TS was described as a severe variant of TS, as discussed below¹ (See FIG. 2).

No neurological sequelae were observed after this episode of cardiac arrest. He later had other arrests, and a cardiac pacemaker was implanted when he was 8 years old. He suffered his most complicated arrest at age 10, which led to a prolonged hospitalization complicated by liver failure, coma, and anoxic brain injury. After this illness, he received an implanted cardioverter-defibrillator (ICD).

His cognitive abilities declined after this episode. He became more socially withdrawn and began attending special education classes with full-time aides. He showed left hemiparesis, which persisted, although not severely. He completed high school and worked for one year as a cleaner at a gym. He has never had a seizure.

At age 19, he had his first episode of major depression, and at 21 he had an episode of mania. Shortly afterwards⁶, TS was described as a mutation in the voltage-gated calcium channel gene CACNA1C. A molecular diagnosis was made when he was 22, and he was treated with a calcium channel blocker (CCB) verapamil at 250 mg/d, which continues to the present time. Theoretically, this would counteract the effects of his mutation, which causes increased Ca⁺⁺ cellular influx during its extended activation⁵. He had a marked decrease in the number of ventricular fibrillation episodes, but no change in QT interval, and continued to have some episodes of a trial fibrillation.

Ten months after starting the medication, he was referred to a neurologist, who found depression and prescribed citalopram. The patient then had significant improvement in mood and behavior.

By age 30 he had experienced 10 manic episodes and 3 hypomanic episodes. These were characterized by periods of up to two weeks duration when his sleep decreased markedly, and he would become aggressive with his siblings and would start fights and verbal altercations that were a departure from his usual behavior. Restlessness, talkativeness and difficult-to-follow speech were also present. Ability to focus was impaired. At age 27, the most extreme manic episode occurred, when, along with the usual symptoms, there were hallucinations and paranoid delusions.

Lithium was then given for mood stabilization, but was not well tolerated, with complaints of increased lethargy and irritability. He was restarted on his antidepressant and has been stable for the three years of observation since then. We note that this is not a typical course of BD, and that further observation will be needed to fully define his course.

Timothy Syndrome (TS) Molecular Pathophysiology

TS is caused by missense mutations in the CACNA1C gene, which encodes the alpha-1 subunit of the L-type calcium channel Caᵥ1.2. All these mutations occur in one of the pore-forming S6 trans-membrane helix segments of the protein. The G406R mutation in exon 8A causes TS1⁶, while TS2 is produced by one of two mutations in the alternate splice form exon 8, G406R or G402S (as in this patient)¹, and a recently reported mutation in exon 38 causes TS3⁷. Our patient is the only known living carrier of the exon 8 G402S mutation, for which he is a mosaic¹. We confirmed his reported mutation by sequencing in our own lab (data not shown).

It is still not completely clear how TS mutations lead to altered function of CaV1.2 channels, but it is known that multiple aspects of channel function are affected. Voltage-dependent inactivation is decreased⁸, action potentials are longer, and calcium flux through Cav1.2 channels is increased⁹⁻¹¹. It is also not known how the delayed inactivation leads to long QT intervals, ventricular fibrillation, or any of the neurological traits associated with the syndrome. In neuronal induced pluripotent stem cells (iPSCs) derived from TS patients, there are multiple changes in gene expression, including increased tyrosine hydroxylase (TH) activity and increased production of norepinephrine and dopamine¹². These changes are most likely related to the role of calcium influx as a second messenger indirectly regulating transcription. Conceivably, these changes in catecholamines are related to the observed arrhythmias and neuropsychiatric changes.

The L-type channel blocker nimodipine failed to reverse excess expression of TH in these neuronal cells¹². However, treatment of the cells with roscovitine, an experimental drug in cancer treatment that inhibits cyclin-dependent kinases and also blocks the calcium channel, specifically enhanced (normalized) inactivation of the L-type channel of the Cav1.2 protein¹³, and caused a 68% reduction in the proportion of TH-positive neurons.

CACNA1C Knockout Mice Cardiac Effects

Two studies of transgenic mice with knockout (KO) of CACNA1C reported opposite effects on heart function. Rosati et al.'s heterozygous KO mouse showed a 58% reduction of CACNA1C mRNA and a 21% reduction in CACNA1C protein, but no change in L-type calcium channel (LTCC) current or in gross cardiac phenotype¹⁴. The Goonasekera study¹⁵ had a graded knockdown heterozygote with cardiac protein levels of CACNA1C reduced by approximately 40%, and roughly 25% less whole-cell LTCC current measured in freshly isolated adult ventricular myocytes. These mice had a pronounced cardiac stress-induced phenotype, but the effects of verapamil were actually detrimental to cardiac function, despite a reduced LTCC current¹⁵. The differences are difficult to resolve. Both studies had a KO cassette maintained on a C57BL/6 background, but there were some differences in the cassette construction. Another mouse study, in which LTCC activity was increased by over-expressing the β2a subunit of the LTCC, found that increased LTCC activity produced a phenotype similar to that of the Goonasekera, leading to cardiac hypertrophy and early death¹⁶. This suggests that it may be a perturbation in calcium influx, rather than specifically an increase or decrease, that leads to a disease phenotype in the mouse models.

CAC1NAC in Bipolar Disorder

The marker most significantly associated with BD in a recent meta-analysis of genome-wide association studies is an intronic SNP within CACNA1C, rs1006737¹⁷. Although the variant with biological effect is not necessarily the associated SNP, it must be in LD with the functional variant, so the biology of the associated SNP is of some interest. One of us (CL) is leading an ongoing study of expression Quantitative Trait Loci (eQTLs) in brain, using the Stanley Medical Research Institute postmortem brain collections. For the current paper, we tested rs1006737 for association with CACNA1C expression levels in human brain tissue.

Materials and Methods for CACNA1C Expression Study

Samples

In our ongoing study, we obtained 164 cerebellum and parietal cortex brain samples from two collections of the Stanley Medical Research Institute (SMRI)¹⁸⁻²⁰. We used the Norgen DNA purification kit to extract high molecular weight DNA from tissue blocks. The DNA was resuspended in low EDTA TE buffer. The DNA concentration and A260/A280 ratio were determined on a NanoDrop ND-1000 spectrophotometer (NanoDrop Technologies, Wilmington, Del.). Only intact DNA samples showing a major band at approximately 10-20 kb on a 1% agarose gel were genotyped.

RNA Preparation and QC

We used the RN easy Mini kit (Qiagen, Valencia, Calif.) to extract total RNA from brain tissue blocks. The ratio of 28S to 18S rRNA and RNA Integrity Number (RIN) were measured using an RNA LabChip kit on the Agilent 2100 Bioanalyzer (Agilent Technologies, Santa Clara, Calif.). To avoid use of seriously degraded samples, only RNA samples with a RIN>7 were used for expression profiling.

Genotyping was performed using Affymetrix GeneChip Mapping 500K Array at TGen by Dr. David Craig (www.tgen.org). Genotypes were called using the BRLMM-p algorithm (Affymetrix). SNPs with call rates ≧99%, Hardy-Weinberg equilibrium (HWE) p values≧0.001 and minor allele frequencies (MAF)≧10% were included in the association tests. Pairwise identity-by-state was calculated using PLINK to verify unrelatedness. We used MACH (http://www.sph.umich.edu/csg/abecasis/MACH/index.html) to impute 852,963 SNPs.

Gene expression profiling was performed using Affymetrix's GeneChip Human Gene 1.0 ST Array, by the NIH Neuroscience Microarray Consortium at Yale University. Raw data in CEL files were summarized by Affymetrix Expression Console software EC1.1, using our customized library files to remove probes that have cross-hybridization to multiple genomic regions, or contain common SNPs. Batch effects were removed by ComBat²¹. All covariates were removed by SVA²² before the SNP-expression association tests.

SNP-expression association tests were performed for cis-regulation. Cis-association refers to correlation of genes and SNPs within 1 Mb of before or after those genes. We used mach2qtl to perform the association tests, and permutation correction. Permutation was used to correct for multiple testing: region-wide significance corrected for the number of SNPs tested; phenotype-wide significance corrected for the number of phenotypes tested as well. No trans-associations (with SNPs outside this region) were found with CACNA1C.

Results of CACNA1C Expression Study

We detect a significant cis-association of CACNA1C expression in cerebellum for SNP rs1006737 that survives correction for region-wide multiple tests, where the risk allele (A) is associated with reduced expression (Table 1). The association is present in multiple exons as well as transcripts of this gene in cerebellum but not in parietal cortex (see Table 2). This finding is also present for rs1024582, the CACNA1C SNP recently associated by Smoller et al.³ with multiple psychiatric disorders. rs1024582 is in nearly complete LD with rs1006737 (R²=0.94), and its risk allele (A) is also associated with significantly decreased cerebellar expression of the same CACNA1C probes (data not shown).

TABLE 1
Association of SNP rs1006737 bipolar risk allele (A) with
decreased expression of CACNA1C in human cerebellum

| Probe | Probe | Frequency | | | | |
|---|---|---|---|---|---|---|
| type | ID | (A allele) | Effect | CHISQ | P-value | Corr_p |
| exon | 7953041 | 0.36 | −0.47 | 14.67 | 1.28E−04 | 0.026 |
| exon | 7953044 | 0.36 | −0.52 | 17.65 | 2.66E−05 | 0.006 |
| exon | 7953049 | 0.36 | −0.61 | 24.46 | 7.59E−07 | 0.001 |
| exon | 7953050 | 0.36 | −0.52 | 17.67 | 2.63E−05 | 0.004 |
| exon | 7953066 | 0.36 | −0.54 | 19.73 | 8.91E−06 | 0.004 |
| exon | 7953068 | 0.36 | −0.55 | 20.18 | 7.04E−06 | 0.002 |
| exon | 7953075 | 0.36 | −0.61 | 24.50 | 7.43E−07 | 0.001 |
| exon | 7953076 | 0.36 | −0.52 | 17.73 | 2.54E−05 | 0.004 |
| exon | 7953079 | 0.36 | −0.50 | 16.30 | 5.41E−05 | 0.014 |
| exon | 7953081 | 0.36 | −0.49 | 16.10 | 6.00E−05 | 0.026 |
| exon | 7953093 | 0.36 | −0.67 | 29.47 | 5.67E−08 | 0.001 |
| exon | 7953095 | 0.36 | −0.55 | 19.84 | 8.43E−06 | 0.004 |
| tran- | 7953040 | 0.36 | −0.60 | 23.73 | 1.11E−06 | 0.001 |
| script | | | | | | |
| tran- | 7953094 | 0.36 | −0.46 | 14.28 | 1.58E−04 | 0.048 |
| script | | | | | | |

Notes:
EFFECT: Beta regression coefficient of expression on SNP after correcting for covariates; CHISQ: Test statistic; P-value for test statistic; Corr_p: regionwide-corrected p-value. Computation by MACHqtl (http://www.sph.umich.edu/csg/abecasis/MACH/download/).

TABLE 2
No association of Bipolar SNP rs1006737 risk allele (A)
with expression of CACNA1C in human parietal cortex

| Probe | Probe | Frequency | | | | |
|---|---|---|---|---|---|---|
| type | ID | (A allele) | Effect | CHISQ | P-value | Corr_p |
| exon | 7953041 | 0.36 | 0.08 | 0.48 | 0.49 | 1 |
| exon | 7953044 | 0.36 | −0.04 | 0.15 | 0.71 | 1 |
| exon | 7953049 | 0.36 | −0.02 | 0.03 | 0.89 | 1 |
| exon | 7953050 | 0.36 | 0.03 | 0.07 | 0.80 | 1 |
| exon | 7953066 | 0.36 | 0.09 | 0.57 | 0.45 | 1 |
| exon | 7953068 | 0.36 | 0.16 | 1.95 | 0.16 | 1 |
| exon | 7953075 | 0.36 | 0.03 | 0.08 | 0.77 | 1 |
| exon | 7953076 | 0.36 | 0.32 | 8.07 | 0.01 | 1 |
| exon | 7953079 | 0.36 | 0.06 | 0.31 | 0.58 | 1 |
| exon | 7953081 | 0.36 | 0.05 | 0.19 | 0.66 | 1 |
| exon | 7953093 | 0.36 | 0.05 | 0.21 | 0.64 | 1 |
| exon | 7953095 | 0.36 | 0.23 | 4.08 | 0.04 | 1 |
| tran- | 7953040 | 0.36 | 0.21 | 3.46 | 0.06 | 1 |
| script | | | | | | |
| tran- | 7953094 | 0.36 | 0.06 | 0.27 | 0.60 | 1 |
| script | | | | | | |

Notes:
The Bipolar disorder risk allele is the minor allele (A), but the analysis program reports on the major allele (G); EFFECT: Beta regression coefficient of expression on SNP after correcting for covariates; CHISQ: Test statistic; P-value for test statistic; Corr_p: regionwide-corrected p-value

Discussion

Cerebellum and Psychiatric Disorders

In a landmark review in 2005, Konarski et al.²³ advanced the paradigm-shifting hypothesis that, based on functional associations evident through cerebellar stimulation, lesions, and functional and morphometric imaging, cerebellar abnormalities play a crucial role in several psychiatric disorders, including schizophrenia, depression, and bipolar disorder. In a 2008 review, Andreasen and Pierson²⁴ remark that “The tentorium was once the Maginot Line of the brain. Supratentorial regions governed “higher cortical functions,” while the humble subtentorial cerebellum performed “lower” functions unrelated to cognition.” They then summarize evidence cortical circuitry connecting the cerebellum and cortex, and conclude that through its role modulating cognition the cerebellum appears to play a crucial role in Schizophrenia. More recent evidence showed, and changes in gene expression of NMDA receptor subunits in cerebellum in Schizophrenia²⁵, and differential expression of genes encoding neuronal ion-channel subunits in cerebellum in several psychiatric disorders including bipolar disorder²⁶. In a whole-genome expression analysis, Chen et al.²⁷ demonstrated two gene co-expression modules associated with Bipolar Disorder and Schizophrenia in multiple data sets. One of the modules was associated with these diagnoses in both cerebral cortex and cerebellum. This module included metallothioneins (MT) and metal binding site functions, which are involved in oxidative stress and other cellular processes. In a related finding, myelination and oxidative stress alterations were observed in the cerebellum of the G72/G30 transgenic schizophrenia mouse model²⁸. In view of all these findings, we would conclude that it is no longer valid to consider the cerebellum as a brain region that cannot play a decisive role in a mental disorder.

Role of Common SNPs in Brain Expression of CACNA1C

It is logical to test for an association between the CACNA1C SNP rs1006737 genotype and CACNA1C expression levels, given the strength and scope of the associations between that SNP and risks of multiple psychiatric diseases, as well as numerous behavioral and cognitive endophenotypes. We report such an association in human cerebellum, but not in parietal cortex. These findings have not yet been replicated in a second dataset; while similar genotype-expression datasets exist, they differ from the current data in multiple ways (See Table 3 for summary). In particular, none included parietal cortex and only one included cerebellum.

TABLE 3
Brain eQTL data: brain regions covered by gene expression and SNP microarrays in different studies

| | | | Gene | | | |
|---|---|---|---|---|---|---|
| | | Brain | expression | | | |
| Authors | N | regions | microarray* | SNP genotype microarray | CB results | PC results |
| Gibbs JR et al.²² | 150 | CB, FC, | GSE15745, | HumanHap550 beadchips, | n.s. | n/a |
| | | PONS, TC | Illumina | 2,545,178 SNPs imputed | | |
| | | | Human Ref-8 | | | |
| | | | v2.0 Expression | | | |
| | | | BeadChip | | | |
| Colantuoni C et | 269 | PFC | GSE30272, | Illumina Infinium II 650K | n/a | n/a |
| al.²⁵ (Bigos et | | | Illumina | or Illumina Infinium HD | | |
| al.²⁴ reported on | | | Human 49K | Gemini 1M Duo | | |
| a subset of data | | | Oligo Array | BeadChips | | |
| from this study). | | | | | | |
| Kang HJ et al.²³ | 57 | 16 brain | GSE25219, | Illumina Omni-2.5 Array | n/a | n/a |
| | | regions | Affymetrix | | | |
| | | | GeneChip | | | |
| | | | Human Exon | | | |
| | | | 1.0 ST Array | | | |
| Liu C et al. (this | 130-150 | PC, CB | Affymetrix | Affymetrix Genome-Wide | rs1006737 associated with | n.s. |
| study) | | | GeneChip | Human SNP Array 500K, | down-regulation of | |
| | | | Human Gene | 852,963 SNPs imputed. | CACNA1C expression | |
| | | | 1.0 ST Array | | | |

Brain regions: PFC prefrontal cortex, PC parietal cortex, CB cerebellum, FC frontal cortex, PFC prefrontal cortex, PONS pons, TC temporal cortex.
*The Affymetrix GeneChip Human Gene 1.0 ST Array probe sets 7953049 and 7953050 are near exons 8 and 8a. Illumina's HumanRef-8 BeadChip has one CACNA1C probe, ILMN_1666775, which maps to a region 187 Kb away from exon 8 and 8a. Illumina's Human 49K Oligo Array has probe HEEBO-098-HCA98N4 near exon 8a. In BrainCloud, the corresponding probe is labeled as 37564. The Affymetrix GeneChip Human Exon 1.0 has two probe sets, 3400806 and 3400807, near exon 8 and 8a.

For the CACNA1C, there is reason to expect different results from different brain regions, because there are differences in relative expression of L-type calcium channels across brain regions. In a mouse model, Schlick et al. found that the ratio of CaV1.2 to CaV1.3 expression was about 1:1 in cortex and hippocampus, while in cerebellum it was 4:1²⁹. Detection of expression differences in CaV 1.2 may thus be more feasible in cerebellum than in cortical regions.

The genome-wide mapping of brain expression and methylation QTLS by Gibbs et al.³⁰ is the one comparable study that included cerebellum. They reported no association between CACNA1C expression and rs1006737 genotype in cerebellum or in the other three regions tested. However, the Illumina expression platform they used had one probe for all of CACNA1C. Our study and Kang et al.'s³¹ QTL mapping of human brain both used an Affymetrix platform with much better CACNA1C coverage. The increased number of probes and regions studied dramatically increased the multiple testing burden in the Kang study, which, combined with the Kang study's small sample size (N=57), left it with low statistical power. None of the probes studied met their criteria for statistical significance of association of SNPs with a gene (gene- wide Bonferroni correction followed by genome-wide Q<0.1) in any of the 16 brain regions they tested, which did not include cerebellum or parietal cortex.

Bigos et al.³² studied the association between CACNA1C genotype and expression in dorsolateral prefrontal cortex, with data that are also included in a later and broader publication by the same group³³. Using another SNP rs2159100 as proxy for rs1006737, since the two are in complete LD (R²=1.0), they reported that that the risk allele is associated with increased CACNA1C expression as measured by probe 28032. To review the group's findings, we used BrainCloud (http://braincloud.jhmi.edu/) to retrieve their cis-eQTL data for CACNA1C. There were actually six expression probes for this gene. Two (28032, 36147) showed nominally significant association with rs2159100 (P=0.022 and 0.028 respectively), with the risk allele (A) having increased expression relative to the non-risk allele. However, the expression probe near exon 8a (37564) and the other three probes were not even nominally associated with rs2159100 genotype. In addition, when the Bonferroni correction for multiple testing is applied (0.05/6 tests=0.008), none of the probes reach gene-wide signficance.

BD in Timothy Syndrome

TS includes a range of neurological, cognitive and psychiatric symptoms, including autism^{1,6,34}, and it would appear from this case that BD is one of them. A possible “confluence of rare/uncommon and common genetic variation on the same genetic [disease] loci” has been noted in GWASs³⁵, which would fit variations in CACNA1C associated with BD. However, we must consider other possibilities: the patient's anoxic brain injury might be considered as an alternative causes. Also, if his symptoms are due to a gain-of-function calcium channel defect, verapamil might be expected to have prevented or treated this defect, as it partially did in his cardiovascular system. Third, BD is a common disease, and could occur in the same patient independently of a rare disease.

Mania can arise after traumatic brain injury (TBI), but has only rarely been reported after anoxic brain injury³⁶. Jorge et al. found a 9.1% incidence of mania after TBI within the first 12 months in 66 patients; but there is little data showing association of mania with a very long interval after head injury, such as the 11 years in this case^{37,38}. Most experts conclude that the longer the latency, the more the attribution of mania to the TBI may be questioned.

If the patient's CACNA1C mutation is responsible for his BD, one might expect that verapamil would also have prevented or treated that condition, as it succeeded in treating his heart condition. However, as noted above, verapamil does not correct the depolarization deficit in TS, or change the intracellular neurotransmitter abnormalities of TS in iPSC cells.¹² Also, the doses used for the management of cardiac conditions are lower than those needed to attain effects in the brain ³⁹, because verapamil has low penetration into the brain, particularly in males

Implications for BD

If the same disease is produced by the TS gain-of-function CACNA1C mutation (increased Ca⁺⁺ flux) as is associated with the common CACNA1C BD risk polymorphism, and there is loss of function from the risk allele of the common polymorphism (decreased Ca⁺⁺ flux from reduced gene expression), this would suggest that Ca⁺⁺ flux that strays in either direction from normal can produce deficits in CNS function in humans, possibly as a result of changes in monoamine neurotransmitter synthesis and release. Our own human brain data, with the largest number of CANA1C expression probes so far examined, demonstrates that individuals with the common polymorphic risk allele have decreased CACNA1C expression in at least one brain region (Table 1). These patients may have a degree of decreased Ca⁺⁺flux due to haploin sufficency, by analogy with the CACNA1C heterozygous graded KO mice of Goonasekera.¹⁵

This would suggest that efficacy of CCB treatment in BD patients would differ in patients with and without the risk allele. Because the CACNA1C risk allele is expected to produce loss of calcium channel function, we would expect BD patients without the risk allele to preferentially respond to CCB treatment. A 2000 review of treatment studies of CCBs in BD⁴¹ concluded that CCBs had not been adequately evaluated as a BD treatment, but evidence that they were generally effective was not present. The reviewed trials had treated BD patients as a single population, but now patient groups can be subdivided according to CACNA1C genotype.

There is already evidence that CACNA1C genotype can affect response to CCBs in treatment of hypertension^{42,43}. A CCB medication which has less of a blood-brain gradient than verapamil, and which, like roscovitine, succeeds in reversing the TH abnormality in TS iPSC cells might be the preferred choice for a clinical trial.

These data indicate that patients with bipolar disorder who do not have the risk allele have increased CACNA1C expression, relative to patients with bipolar disorder who have one or two copies of the risk allele. Accordingly, these data indicate that patients with bipolar disorder who do not have the risk allele respond better to treatment with calcium channel blocking drugs.

### Example 2

This example demonstrates a clinical study on the use of calcium channel blockers (CCB) as an augmentation therapy for patients with mania (including schizoaffective manic patients).

Response to CCB as an augmentation therapy is tested in a series of 90 patients (total for three collaborating centers). Bipolar I or Schizoaffective-manic patients between ages 18 and 50, who are not currently treated with a CCB, do not have a medical contraindication to CCB treatment, and are currently manic and hospitalized, are treated with CCB as an add-on to Treatment As Usual (TAU). Each patient is followed until mania ratings have subsided to <4 on YMRS or two weeks have passed. At the end of the study of 90 patients, genome-wide association genotyping is performed on all patients, and calcium channel gene polymorphisms are tested for correlation with clinical response to CCB treatment.

Patients for the study are selected as follows:

Clinical evaluation: At the time of hospital admission or as soon as possible afterward, the patient is invited to consent to the study. They then have a baseline examination to confirm the diagnosis of acute mania, and to obtain a medication history. This includes a clinical examination, review of medical records and treatment history, and interview with relatives and any significant other person who may know the patient's history.

Mania is diagnosed by satisfying DSM-V criteria for a manic epdisode (DSM-V Bipolar I description), Young Mania Rating Scale [7] (YMRS) score >15, and lifetime history of at least one previous episode of mania (based on medical records or history provided by a reliable source).

The criteria for excluding an individual from the study are as follows:
1) Unwilling or unable to comply with study requirements.
2) History of CCB-related toxicity or hypersensitivity.
3) Currently treated with a CCB.
3) Previous cardiac surgery. Previous diagnosis of certain cardiac (heart) or vascular disorders, including cardiac arrhythmia (heart rhythm problem), atherosclerosis (blockage of blood vessels), congestive heart failure, myocardial infarction, angina, cerebrovascular disease.
4) High blood pressure, renal failure.
5) Clinically significant hypotension (low blood pressure)
6) Women who are pregnant, breastfeeding, or of child-bearing potential and aren't able to agree to medically acceptable contraception.
7) Currently active substance abuse or dependence.

The following treatment protocol is followed:

Prior to entering the treatment protocol, each participant receives an EKG and comprehensive metabolic panel. Patients with evidence of clinically significant abnormalities in cardiac, hepatic, or renal function, based on these tests or other clinical examination, are excluded.

Treatment as Usual: The guidelines for treatment as usual (TAU) are well accepted [8], and indicate that the foundation of treatment as usual is to maintain treatment with at least one FDA approved mood stabilizer (including Lithium, certain anticonvulsants, and certain atypical antipsychotics) and to follow the recommendations summarized in the evidence-based stages of Texas Implementation of Medication Algorithm (TIMA) revised guidelines. For all patients in this protocol, TAU requires the presence of at least one FDA-approved mood stabilizer and antidepressant medications are only prescribed in combination with a mood stabilizing drug, as described in the TIMA guidelines.

All participants receive TAU for manic episode bipolar or schizoaffective disorder [8] either provided by the principal investigator or by co-investigator clinical psychiatrists or psychiatry residents trained and supervised in optimal treatment practices for bipolar disorder within inpatient hospital settings.

CCB treatment: Nicardipine satisfactorily crosses the blood-brain barrier, and has a long history of clinical use for cardiac arrhythmias, hypertension, and Reynaud phenomenon. The initial dosage is 20 mg po three times daily (60 mg per day). After 3 days, re-evaluation of dosage is made. If clinical state of mania has not improved, and there is not significant reduction in resting and standing blood pressure, dosage is increased to up to 120 mg per day.

Patient ratings: CGI-S-BP for Mania and for Depression and Young Mania Rating Scale are completed daily by the treating psychiatrist or by a designated collaborating resident or other professionally qualified trained rater.

Genetic test: The PsychChip under development by Illumina for genome-wide association studies has over 200,000 common SNPs on it, including the CACNA1C risk SNPs and all the SNPs of calcium channel genes that have suggestive association in the literature or have been of interest in psychiatric disorders. All patients with clinical data on their manic episode are genotyped using this PscyhChip.

Response analysis: The endpoint is either scored on YMRS <4 or 2 weeks in the trial [9]. Survival analysis is performed using the Kaplan-Meier method [10] for the main hypothesis of CACNA1C risk allele. For an exploratory analysis, the Cox proportional hazards model [11] is used, incorporating as covariates all genotypes of Calcium channel genes with p<0.001 in meta-analyes of genome-wide association tests of Bipolar disorder.

Statistical power: This is a preliminary trial, and we may not have enough data on treatment response to estimate power to discriminate two groups of patients.

Risks/Benefits: The potential benefit of this protocol is improved treatment of mania, a disorder that can be life-threatening to the patient because of risky behavior and because of manic exhaustion, and is difficult to manage with current medication. Common side effects of nicardipine include headache, peripheral edema, dizziness, flushing, asthenia, angina, hypotension, nausea/vomiting, tachycardia, and palpitations. Serious reactions include angina exacerbation, AV block, myocardial infarction, pericarditis, ventricular tachycardia, deep vein thrombosis, thrombocytopenia, and hypersensitivity reaction. The probability of serious reactions is minimized by not including patients with history of cardiovascular disorder or with abnormal measured risk factors for cardiovascular disorder, such as extreme obesity or abnormal LDL cholesterol. Patients with compromised renal or hepatic function, which can be associated with adverse effects of nicardipine, are excluded from the study.

Clinical Evaluation Protocol:

Symptom Severity

Each day, symptom raters examine patient and review nursing and medical records. Based on all this information, mania is rated using CGI-S-BP for Mania and for Depression, and the Young Mania Rating Scale (implemented in integrated version with CARS-M (see below)). The raters are attending or resident doctors on the inpatient service, nurses, or research assistants.

Clinical Global Impressions of Severity Scale-Bipolar Version (CGI-BP) [12]. The CGI-BP is a modified version of the original CGI designed specifically for use in assessing global illness severity and/or change in patients with bipolar disorder and assesses overall bipolar illness, depression, and mania. While the original CGI has been criticized for lack of reliability, the CGI-BP has been shown to have excellent inter-rater reliability [12]. Not surprisingly, placebo response rates have been shown to be lower with the CGI-BP, compared to the HAM-D or MADRS in bipolar disorder [13]. In contrast to symptom-severity scales, the CGI-BP is an integrated measure of illness severity. In contrast to the MADRS and HAM-D, it is not encumbered by the inability to distinguish improved somatic function (appetite and sleep) from medication-induced adverse events, which is important when studying medications that cause weight gain or somnolence.

Young Mania Rating Scale (YMRS). An 11-item, clinician-rated measure that queries symptoms of mania.

Clinician Administered Rating Scale for Mania (CARS-M) [14]. The CARS-M is a reliable and valid 15 item, clinician rated measure of mania The CARS-M incorporates a number of methodological improvements in comparison to more frequently utilized mania rating scales, such as the YMRS. For example, the CARS-M separately assesses the presence of psychotic symptoms (e.g., delusions and hallucinations). Given the overlap in symptoms assessed on the YMRS and CARS-M, an integrated version will be developed and utilized for the current study, minimizing patient burden, yet allowing full scale scores to be derived for each measure.

When it is clinically feasible, patients undergo a detailed interview scale (Diagnostic Interview for Genetic Studies (DIGS). Data are collected on course of illness including age of onset for bipolar disorder, number of prior episodes, past treatment response, childhood abuse (emotional, physical, sexual), medical conditions, psychoactive substance use, and family history, prior treatment and prior suicide attempt history, including lethality.

The following references are cited in the above example.
1. Gershon E S, Grennan K, Busnello J, Badner J A, Ovsiew F, Memon S et al.: A rare mutation of CACNA1C in a patient with bipolar disorder, and decreased gene expression associated with a bipolar-associated common SNP of CACNA1C in brain. Mol Psychiatry 19(8):890-4 (2013).
2. Caillard V, Masse G: Treatment of mania by a calcium inhibitor. Preliminary study. Encephale 1982, 8: 587-594.
3. Casamassima F, Hay A C, Benedetti A, Lattanzi L, Cassano G B, Perlis RH: L-type calcium channels and psychiatric disorders: A brief review. Am J Med Genet B Neuropsychiatr Genet 2010, 153B: 1373-1390.
4. Pazzaglia P J, Post R M, Ketter T A, Callahan A M, Marangell L B, Frye M A et al.: Nimodipine monotherapy and carbamazepine augmentation in patients with refractory recurrent affective illness. J Clin Psychopharmacol 1998, 18: 404-413.
5. Bachmeier C, Beaulieu-Abdelahad D, Mullan M, Paris D: Selective dihydropyiridine compounds facilitate the clearance of beta-amyloid across the blood-brain barrier. Eur J Pharmacol 2011, 659: 124-129.
6. Paris D, Bachmeier C, Patel N, Quadros A, Volmar C H, Laporte V et al.: Selective antihypertensive dihydropyridines lower Abeta accumulation by targeting both the production and the clearance of Abeta across the blood-brain barrier. Mol Med 2011, 17: 149-162.
7. Young R C, Biggs J T, Ziegler V E, Meyer D A: A rating scale for mania: reliability, validity and sensitivity. Br J Psychiatry 1978, 133: 429-435.
8. Suppes T, Dennehy E B, Hirschfeld R M, Altshuler L L, Bowden C L, Calabrese J R et al.: The Texas implementation of medication algorithms: update to the algorithms for treatment of bipolar I disorder. J Clin Psychiatry 2005, 66: 870-886.
9. Berk M, Ng F, Wang W V, Calabrese J R, Mitchell P B, Malhi G S et al.: The empirical redefinition of the psychometric criteria for remission in bipolar disorder. J Affect Disord 2008, 106: 153-158.
10. Kaplan E L, Meier P: Nonparametric Estimation from Incomplete Observations. Journal of the American Statistical Association 1958, 53: 457-481.
11. Cox D R, Oakes D: Analysis of Survival Data. Chapman & Hall; 1998.
12. Spearing M K, Post R M, Leverich G S, Brandt D, Nolen W: Modification of the Clinical Global Impressions (CGI) Scale for use in bipolar illness (BP): the CGI-BP. Psychiatry Res 1997, 73: 159-171.
13. Calabrese J R, Bowden C L, Sachs G S, Ascher J A, Monaghan E, Rudd G D: A double-blind placebo-controlled study of lamotrigine monotherapy in outpatients with bipolar I depression. Lamictal 602 Study Group. J Clin Psychiatry 1999, 60: 79-88.
14. Altman E G, Hedeker D R, Janicak P G, Peterson J L, Davis J M: The Clinician-Administered Rating Scale for Mania (CARS-M): development, reliability, and validity. Biol Psychiatry 1994, 36: 124-134.

### Example 3

This example demonstrates the testing of additional SNPs.

SNPs in Table A are tested for predictive power of response to CCB treatment in patients undergoing the protocol in Example 2, and in other clinical protocols studying treatment response in other affective disorder diagnoses, which are described above in the section entitled “Mood Disorders.”

### Example 4

This example demonstrates a method of determining a treatment regimen for a subject with a mood disorder.

DNA is extracted from whole blood of the subject using known methods. Presence of allele [A] is determined by Sanger sequencing, or by microarray methods, or by PscyhChip, or other known DNA polymorphism detection methods. If Sanger sequencing is used, the primers used are: Forward primer: CCACTTGGCTCTATCAAAGTCT (SEQ ID NO: 50) and Reverse primer: CCTGAGAGACACTGTGAGG (SEQ ID NO: 51). This amplifies a fragment of 100 bp, which contains the SNP rs1006737. Sequencing result can determine whether the subject has A or G allele. Subjects that have one or fewer copies of allele [A] of CACNA1C are treated with a calcium channel blocker.

The following represents a list of references cited in Example 1.

1. Splawski I, et al. Severe arrhythmia disorder caused by cardiac L-type calcium channel mutations. Proceedings of the National Academy of Sciences of the United States of America. 2005; 102:8089-8096.

2. Ferreira M A, et al. Collaborative genome-wide association analysis supports a role for ANK3 and CACNA1C in bipolar disorder. Nat Genet. 2008

3. Cross-Disorder Group of the Psychiatric Genomics Consortium Identification of risk loci with shared effects on five major psychiatric disorders: a genome-wide analysis. Lancet. 2013

4. Psychiatric GWAS Consortium Bipolar Disorder Working Group et al. Large-scale genome-wide association analysis of bipolar disorder identifies a new susceptibility locus near ODZ4. Nat Genet. 2011

5. Jacobs A, Knight B P, McDonald K T, Burke M C. Verapamil decreases ventricular tachyarrhythmias in a patient with Timothy syndrome (LQT8) Heart Rhythm. 2006; 3:967-970.

6. Splawski I, et al. Ca(V)1.2 calcium channel dysfunction causes a multisystem disorder including arrhythmia and autism. Proc Natl Acad Sci USA. 2004; 119:19-31.

7. Gillis J, et al. Long Q T, syndactyly, joint contractures, stroke and novel CACNA1C mutation: Expanding the spectrum of Timothy syndrome. Am J Med Genet A. 2012; 158A:182-187.

8. Barrett C F, Tsien R W. The Timothy syndrome mutation differentially affects voltage- and calcium-dependent inactivation of CaV1.2 L-type calcium channels. 2008; 105:2157-2162.

9. Thiel W H, et al. Proarrhythmic defects in Timothy syndrome require calmodulin kinase II. Circulation. 2008; 118:2225-2234.

10. Erxleben C, et al. Cyclosporin and Timothy syndrome increase mode 2 gating of CaV1.2 calcium channels through aberrant phosphorylation of S6 helices. Proceedings of the National Academy of Sciences of the United States of America. 2006; 103:3932-3937.

11. Depil K, et al. Timothy mutation disrupts the link between activation and inactivation in Ca(V)1.2 protein. J Biol Chem. 2011; 286:31557-31564.

12. Pasca S P, et al. Using iPSC-derived neurons to uncover cellular phenotypes associated with Timothy syndrome. Nat Med. 2011; 17:1657-1662.

13. Yarotskyy V, Gao G, Peterson B Z, Elmslie K S. The Timothy syndrome mutation of cardiac CaV1.2 (L-type) channels: multiple altered gating mechanisms and pharmacological restoration of inactivation. J Physiol. 2009; 587:551-565.

14. Rosati B, et al. Robust L-type calcium current expression following heterozygous knockout of the Cav1.2 gene in adult mouse heart. J Physiol. 2011; 589:3275-3288.

15. Goonasekera S A, et al. Decreased cardiac L-type Ca(2)(+) channel activity induces hypertrophy and heart failure in mice. J Clin Invest. 2012; 122:280-290.

16. Nakayama H, et al. Ca2+- and mitochondrial-dependent cardiomyocyte necrosis as a primary mediator of heart failure. J Clin Invest. 2007; 117:2431-2444.

17. Liu Y, et al. Meta-analysis of genome-wide association data of bipolar disorder and major depressive disorder. Mol Psychiatry. 2011; 16:2-4.

18. Knable M B, Barci B M, Webster M J, Meador-Woodruff J, Torrey E F. Molecular abnormalities of the hippocampus in severe psychiatric illness: postmortem findings from the Stanley Neuropathology Consortium. Mol Psychiatry. 2004; 9544:609-20.

19. Torrey E F, Webster M, Knable M, Johnston N, Yolken R H. The stanley foundation brain collection and neuropathology consortium. Schizophr Res. 2000; 44:151-155.

20. Torrey E F, et al. Neurochemical markers for schizophrenia, bipolar disorder, and major depression in postmortem brains. Biol Psychiatry. 2005; 57:252-260.

21. Johnson W E, Li C, Rabinovic A. Adjusting batch effects in microarray expression data using empirical Bayes methods. Biostatistics. 2007; 8:118-127.

22. Leek J T, Storey J D. Capturing heterogeneity in gene expression studies by surrogate variable analysis. PLoS Genet. 2007; 3:1724-1735.

23. Konarski J Z, McIntyre R S, Grupp L A, Kennedy S H. Is the cerebellum relevant in the circuitry of neuropsychiatric disorders? J Psychiatry Neurosci. 2005; 30:178-186.

24. Andreasen N C, Pierson R. The role of the cerebellum in schizophrenia. Biol Psychiatry. 2008; 64:81-88.

25. Schmitt A, et al. Gene expression of NMDA receptor subunits in the cerebellum of elderly patients with schizophrenia. Eur Arch Psychiatry Clin Neurosci. 2010; 260:101-111.

26. Smolin B, Karry R, Gal-Ben-Ari S, Ben-Shachar D. Differential expression of genes encoding neuronal ion-channel subunits in major depression, bipolar disorder and schizophrenia: implications for pathophysiology. Int J Neuropsychopharmacol. 2012; 15:869-882.

27. Chen C, et al. Two Gene Co-expression Modules Differentiate Psychotics and Controls. Molecular Psychiatry. 2012 Ref Type: In Press.

28. Filiou M D, Teplytska L, Otte D M, Zimmer A, Turck C W. Myelination and oxidative stress alterations in the cerebellum of the G72/G30 transgenic schizophrenia mouse model. J Psychiatr Res. 2012; 46:1359-1365.

29. Schlick B, Flucher B E, Obermair G J. Voltage-activated calcium channel expression profiles in mouse brain and cultured hippocampal neurons. Neuroscience. 2010; 167:786-798.

30. Gibbs J R, et al. Abundant quantitative trait loci exist for DNA methylation and gene expression in human brain. PLoS Genet. 2010; 6:e1000952.

31. Kang H J, et al. Spatio-temporal transcriptome of the human brain. Nature. 2011; 478:483-489.

32. Bigos K L, et al. Genetic variation in CACNA1C affects brain circuitries related to mental illness. Arch Gen Psychiatry. 2010; 67:939-945.

33. Colantuoni C, et al. Temporal dynamics and genetic control of transcription in the human prefrontal cortex. Nature. 2011; 478:519-523.

34. Bader P L, et al. Mouse model of Timothy syndrome recapitulates triad of autistic traits. Proceedings of the National Academy of Sciences of the United States of America. 2011; 108:15432-15437.

35. Panagiotou O A, Evangelou E, Ioannidis J P. Genome-wide significant associations for variants with minor allele frequency of 5% or less—an overview: A HuGE review. Am J Epidemiol. 2010; 172:869-889.

36. Calache M J, Bourgeois M. Bipolar affective disorder and anoxic brain damage. Br J Psychiatry. 1990; 157:458-459.

37. Jorge R, Robinson R G. Mood disorders following traumatic brain injury. Int Rev Psychiatry. 2003; 15:317-327.

38. Shukla D, Devi B I, Agrawal A. Outcome measures for traumatic brain injury. Clin Neurol Neurosurg. 2011; 113:435-441.

39. May A. The importance of the heart in cluster headache treatment. Nat Clin Pract Neurol. 2008;4:182-183.

40. van Assema D M, et al. P-glycoprotein function at the blood-brain barrier: effects of age and gender. Mol Imaging Biol. 2012; 14:771-776.

41. Levy N A, Janicak P G. Calcium channel antagonists for the treatment of bipolar disorder. Bipolar Disord. 2000; 2:108-119.

42. Kamide K, et al. Genetic polymorphisms of L-type calcium channel alpha1C and alpha1D subunit genes are associated with sensitivity to the antihypertensive effects of L-type dihydropyridine calcium-channel blockers. Circ J. 2009; 73:732-740.

43. Bremer T, Man A, Kask K, Diamond C. CACNA1C polymorphisms are associated with the efficacy of calcium channel blockers in the treatment of hypertension. Pharmacogenomics. 2006; 7:271-279

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating a mood disorder in a subject, comprising
administering to the subject a calcium channel blocker (CCB) when no more than one copy of allele [A] is present in the cells of the sample;
wherein the method comprises the step of analyzing a sample obtained from the subject for the presence of an allele [A] of CACNA1C, wherein allele [A] comprises the sequence of polymorphic marker rs1006737 before the step of administering the CCB,
or
wherein the subject is a subject from which a sample was obtained, wherein the copy number of allele [A] of CACNA1C, comprising the sequence of polymorphic marker rs1006737, in the sample has been analyzed.

2. (canceled)

3. A method of determining a treatment regimen for a subject with a mood disorder, comprising:
a) analyzing a sample obtained from a subject with a mood disorder for the presence of allele [A] of CACNA1C, wherein allele [A] comprises the sequence of the polymorphic marker rs1006737; and
b) selecting a treatment regimen comprising administration of a calcium channel blocker (CCB), when no more than one copy of allele [A] is present in the cells of the sample.

4. The method of claim 1, wherein the subject is heterozygous for allele [A].

5. (canceled)

6. The method of claim 1, comprising administering to the subject a CCB when allele [A] is absent from the cells of the sample, and optionally, comprising selecting a treatment regimen comprising administration of a CCB, when allele [A] is absent from the sample.

7. (canceled)

8. The method of claim 1, wherein the CCB is selected from the group consisting of amlodipine, diltiazem, felodipine, isradipine, nicardipine, nifedipine, nisoldipine and verapamil.

9. (canceled)

10. The method of claim 1, wherein the mood disorder is a depressive disorder, optionally, a bipolar disorder.

11. (canceled)

12. The method of claim 10, wherein the bipolar disorder is bipolar I, bipolar II, cyclothymia, or biopolar disorder not otherwise specified.

13. The method of claim 1, wherein the sample comprises DNA from a blood cell of the subject or comprises DNA from the saliva of the subject.

14. (canceled)

15. The method of claim 1, further comprising analyzing the sample for CACNA1C expression.

16. The method of claim 1, further comprising genotyping the sample for one or more of the polymorphic markers listed in Table A.

17. The method of claim 1, further comprising administering a therapeutic compound other than a CCB.

18. The method of claim 17, wherein the therapeutic compound is a mood stabilizer.

19. The method of claim 18, wherein the mood stabilizer is lithium, an anticonvulsant, or an atypical antipsychotic.

20. The method of claim 3, wherein the treatment regimen further comprises administration of a therapeutic compound other than a CCB.

21. The method of claim 20, wherein the therapeutic compound is a mood stabilizer.

22. The method of claim 21, wherein the mood stabilizer is lithium, an anticonvulsant, or an atypical antipsychotic.

23.-26. (canceled)

27. A kit comprising a nucleic acid molecule which hybridizes to or specifically binds to a region of SEQ ID NO: 1 which is about 20 basepairs (bp) to about 1000 bp upstream or downstream of position 270344 of SEQ ID NO: 1.

28. The kit of claim 27, comprising a nucleic acid molecule of SEQ ID NO: 50 and a nucleic acid molecule of SEQ ID NO: 51.
